(19) United States
(12) Patent Application Publication
(10) Pub. No.: US 2011/0112167 A1
(43) Pub. Date: May 12, 2011

(54) THERAPEUTIC AGENTS AND TARGETS

(75) Inventors: Gregory Royce Collier, Barwon Heads (AU); Kenneth Russell Walder, Ocean Grove (AU); Janine Susan McMillan, Torquay (AU); David Harry Segal, Ocean Grove (AU); Kelly Fiona Windmill, Newtown (AU); Kristy Ann Bolton, Geelong West (AU)

(73) Assignee: AUTOGEN RESEARCH PTY LTD, Brighton (AU)

(21) Appl. No.: 11/914,521

(22) PCT Filed: May 17, 2006
(86) PCT No.: PCT/AU06/00659

§ 371 Date: Jul. 8, 2008

Related U.S. Application Data
(60) Provisional application No. 60/681,889, filed on May 17, 2005.

(51) Int. Cl. A61K 31/7088; C12Q 1/68; C12N 15/11

(52) U.S. Cl. 514/44 A; 435/6; 536/23.5

(57) ABSTRACT

The present invention relates to diagnostic and therapeutic methods in relation to diabetic complications, such as blindness, nephropathy and cardiovascular disease, and inflammatory conditions, such as angina, arthritis, empyema pharyngitis and urinary tract infection. Diagnostic methods involve screening for up regulated expression of decor (Den) or thioredoxin-like protein 19 (TLP 19). Therapeutic methods involve modulation of expression or activity o Den or TLP 19. The invention also relates to screening methods for identifying functional signal sequences to screen for secreted, membrane-bound and exported proteins and cell surface receptors.

### BACKGROUND OF THE INVENTION

1. Field of the Invention

The present invention relates generally to the field of therapeutic agents and targets. More particularly, the present invention employs the identification of functional signal sequences to screen for secreted and membrane-bound targets, exported proteinaceous molecules and cell surface receptors. The present invention further extends to the use of these molecules or antagonists or agonists thereof in medical treatment and/or diagnostic protocols. The present invention further contemplates a screening protocol for potential therapeutic targets.

2. Description of the Prior Art

Bibliographic details of references provided in the subject specification are listed at the end of the specification.

Reference to any prior art in this specification is not, and should not be taken as, an acknowledgment or any form of suggestion that this prior art forms part of the common general knowledge in any country.

The increasing sophistication of recombinant DNA technology is greatly facilitating research and development in the veterinary and allied human and animal health fields. This is particularly the case in the investigation of the genetic bases involved in the etiology of certain disease conditions. Diseases of particular concern include disorders associated with diabetes, complications associated with diabetes, such as blindness, nephropathy and/or cardiovascular disease, and mitochondrial dysfunction as well as myopathies, genetic disorders and cancers and in modulating apoptosis, signal transduction and/or nuclear targeting.

Diabetes represents a significant and debilitating disease. The incidence of diabetes is increasing rapidly. It has been estimated that there were about 700,000 persons with diabetes in Australia in 1995 while in the US, the prevalence of diabetes increased from 4.9% in 1990 to 6.9% in 1999 (Mokdad, Diabetes Care 24 (2): 412, 2001).

There are two main types of diabetes referred to as Type 1 and Type 2 diabetes.

Type 1 diabetes, also known as insulin-dependent diabetes mellitus (IDDM), results from an inability to produce insulin. It can develop at any age, although it usually develops in children and young adults and is also referred to as juvenile-onset diabetes. Once it has developed, Type 1 diabetes is a life-long condition.

Type 2 diabetes occurs later in life and is sometimes known as late-onset diabetes or non-insulin-dependent diabetes mellitus (NIDDM), because insulin treatment is not always needed. Type 2 diabetes develops when the body becomes resistant to insulin. This happens when the body's tissues, such as muscle, do not respond fully to the actions of insulin, so cannot make use of glucose in the blood. The pancreas responds by producing more insulin. In addition, the liver, where glucose is stored, releases more glucose to try to increase the amount of glucose available. Eventually, the pancreas becomes less able to produce enough insulin and the tissues become more resistant to insulin. As a result, blood glucose levels slowly start to rise.

Mitochondrial dysfunction refers to any illness resulting from a deficiency of any mitochondrial-located protein which is involved in energy metabolism. Therefore, deficiencies of the respiratory (electron transport) chain, either resulting from a deficiency in one or more of the mitochondrial or nuclear-encoded proteins, are mitochondrial disorders. Also, by definition, disorders of the fatty acid (beta) oxidation, Krebs cycle and pyruvate dehydrogenase complex deficiency are mitochondrial disorders. Although these disorders may be genetically dissimilar, mitochondrial dysfunction results in an energy deficient state.

There is no one identifying feature of mitochondrial disease. Subjects can have combinations of problems whose onset may occur from before birth to late adult life. Mitochondrial diseases should be considered in the differential diagnosis when there are unexplained features, especially when these occur in combination. Mitochondrial disease and disorders can affect multiple organs, resulting in a vast array of symptoms. Symptoms which may affect the brain include, developmental delays, mental retardation, dementia, seizures, neuro-psychiatric disturbances, atypical cerebral palsy, migraines, strokes.

Cancer is also one of the most debilitating disease conditions affecting predominantly humans but also a range of animals. The health cost to the world-wide community runs into the billions of dollars, let alone the personal cost to families.

Diabetes, mitochondrial disease and cancer, therefore, are significant conditions requiring expenditure of time and financial resources to develop new methods of treatment, prevention and diagnosis.

In International Patent Application No. PCT/AU02/00109 [WO 02/062994] which is incorporated herein by reference, techniques including differential display analysis and macroarray (i.e. membrane-based microarray) analysis of genetic material from hypothalamus tissue or muscle tissue were used to identify candidate genetic sequences associated with a healthy state or with physiological conditions such as obesity, anorexia, weight maintenance, diabetes, muscle development and/or metabolic energy levels. An animal model was employed comprising the Israeli Sand Rat (Psammomys obesus). Three groups of animals are used designated Groups A, B and C based on metabolic phenotype as follows:—

Group A: lean animals;
Group B: obese, non-diabetic animals; and
Group C: obese, diabetic animals.

Animals were maintained under two study conditions: (1) they were either fed ad libitum (“fed”) or fasted for 24 hours (“fasted”) prior to analysis; or (2) maintained by being fed ad libitum (“control”) or placed on an energy restricted diet (“restricted”), and genetic sequences analyzed by microarray analysis.

Alternative techniques are required to identify potential therapeutic agents and targets.

Cell surface receptors for hormones and other intercellular signalling molecules represent excellent candidates for the development of novel therapeutics. These proteins usually possess a short hydrophobic domain in the N-terminus of the protein (a signal sequence) which allows the nascent polypeptide to enter the endoplasmic reticulum during translation. Bioinformatic algorithms have been developed to identify these genes by predicting the presence or absence of signal peptides from N-terminal amino acid sequence signatures of known genes. These algorithms although being powerful tools, will not identify genes that lack a conventional signal peptide or a full-length coding sequence.

In accordance with the present invention, a protocol is developed to identify signal sequences which in turn are used to identify exported or cell surface molecules involved in cell signalling. Such molecules are proposed to be useful therapeutic agents and/or targets. The genes encoding these molecules will be used in microarray analysis to identify genes encoding secreted and/or membrane bound proteins that are differentially expressed in and associated with subjects with inter alia obesity, insulin resistance, type 2 diabetes and inflammation.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

A summary of genes identified in accordance with the present invention is provided in Table 1.

Nucleotide and amino acid sequences are referred to by a sequence identifier number (SEQ ID NO:). The SEQ ID NO: correspond numerically to the sequence identifiers <400>1 (SEQ ID NO:1), <400>2 (SEQ ID NO:2), etc. A summary of the sequence identifiers is provided in Table 2. A sequence listing is provided after the claims.

All scientific citations, patents, patent applications and manufacturer's technical specifications referred to hereinafter are incorporated herein by reference in their entirety.

A protocol referred to as a “Signal Sequence Trap” (“SST”) is developed to identify genes encoding proteins which are secreted or expressed on the cell surface. This approach has the advantage of not requiring full sequence information of target genes and is capable of identifying genes which encode a functional signal peptide in any tissue or cell line of interest. Once identified, positive SST cDNAs are screened by microarray for differential expression.

A 5′-enriched Psammomys obesus skeletal muscle cDNA library is generated and inserted upstream of signal sequence-less IL-3 in a retroviral vector. This is transfected into Plat-E cells and the virus produced is used to infect the IL-3 dependent cell line FDCP1 cells. Only cells secreting IL-3 (which contain a clone with a functional signal sequence) survive. Over 1200 positive clones were generated, the DNA extracted, and the insert amplified by PCR and spotted onto microarray slides. The expression of these genes in an animal model of obesity and diabetes has been determined using microarray analysis.

cDNA microarray technology provides a powerful technical means to generate a gene expression database of both known genes and unknown transcripts. Using cDNA microarrays, comparative estimates can be obtained of the level of gene expression of large numbers of genes (up to 20,000 per microarray) in each sample. cDNA microarrays generally involve a large number of DNA “spots” in an orderly array chemically coupled to the surface of a solid substrate, usually but not exclusively an optically flat glass microscope slide. Fluorescently labeled cDNAs are generated from experimental and reference RNA samples and then competitively hybridized to the gene chip. The experimental and reference cDNAs are labeled with a different fluorescent dye and the intensity of each fluor at each DNA spot gives an indication of the level of that particular RNA species in the experimental sample relative to the reference RNA. The ratio of fluorescence can be taken as a measure of the expression level of the gene corresponding to that spot in the experimental sample.

In a preferred embodiment, six expressed sequences exhibiting signal sequence properties have been identified from P. obesus designated herein CXS-740 [SEQ ID NO:1], CXS-741 [SEQ ID NO:2], CXS-742 [SEQ ID NO:3], CXS-743 [SEQ ID NO:4], CXS-744 [SEQ ID NO:5] and CXS-745 [SEQ ID NO:6].

A summary of the CXS genes is provided in Table 1.

The present invention contemplates the use of these sequences or mammalian including human homologs thereof or their expression products in the manufacture of medicaments and diagnostic agents for a range of metabolic conditions such as Type I or Type II diabetes, inflammation, mitochondrial dysfunction, myopathy, genetic disorders, energy imbalance and/or obesity as well as to modulate apoptosis, signal transduction and/or nuclear targeting.

The present invention provides, therefore, a nucleic acid molecule comprising a sequence of nucleotides encoding or complementary to a sequence encoding an expression product or a derivative, homolog, analog or mimetic thereof wherein said nucleic acid molecule or its homolog is said expression product identified as being a secreted or cell surface molecule and involved in metabolic signalling in a cell or group of cells.

More particularly, the present invention provides a nucleic acid molecule comprising a nucleotide sequence encoding or complementary to a sequence encoding an expression product or a derivative, homolog, analog or mimetic thereof wherein the nucleotide sequence is as substantially set forth in SEQ ID NO:1 (CXS-740) or SEQ ID NO:2 (CXS-741) or SEQ ID NO:3 (CXS-742) or SEQ ID NO:4 (CXS-743) or SEQ ID NO:5 (CXS-744) or SEQ ID NO:6 (CXS-745) or a nucleotide sequence having at least about 50% similarity to all or part of SEQ ID NO:1 or SEQ ID NO:2 or SEQ ID NO:3 or SEQ ID NO:4 or SEQ ID NO:5 or SEQ ID NO:6 and/or is capable of hybridizing to one or more of SEQ ID NO:1 or SEQ ID NO:2 or SEQ ID NO:3 or SEQ ID NO:4 or SEQ ID NO:5 or SEQ ID NO:6 or a complementary form thereof under low stringency conditions at 42° C.

Even more particularly, the present invention identifies CXS-741 (SEQ ID NO:2) and CXS-744 (SEQ ID NO:5) as being elevated in biological samples such as fluid samples from subjects with diabetes, complications of diabetes and inflammation, hence providing diagnostic and therapeutic targets.

Accordingly, another aspect of the present invention contemplates a method for the prognosis of diabetes or a complication arising from diabetes in a subject, said method comprising screening for elevated levels of CXS-741 (SEQ ID NO:2) and/or CXS-744 (SEQ ID NO:5) protein or mRNA encoding said protein or a homolog thereof in a biological sample from said subject wherein an elevated level is indicative of diabetes or a complication arising therefrom or a likelihood of development of same.

Still another aspect of the present invention provides a method for the prognosis of an inflammatory condition in a subject, said method comprising screening for elevated levels of CXS-741 (SEQ ID NO:2) and/or CXS-744 (SEQ ID NO:5) protein or mRNA encoding said protein or a homolog thereof in a biological sample from said subject wherein an elevated level is indicative of an inflammatory condition.

The present invention also provides an isolated expression product or a derivative, homolog, analog or mimetic thereof which expression product is encoded by a nucleotide sequence said expression product identified as being a secreted or cell surface molecule and involved in metabolic signalling in a cell or group of cells.

More particularly, the present invention is directed to an isolated expression product or a derivative, homolog, analog or mimetic thereof wherein the expression product is encoded by a nucleotide sequence substantially as set forth in SEQ ID NO:1 (CXS-740) or SEQ ID NO:2 (CXS-741) or SEQ ID NO:3 (CXS-742) or SEQ ID NO:4 (CXS-743) or SEQ ID NO:5 (CXS-744) or SEQ ID NO:6 (CXS-745) or a nucleotide sequence having at least 50% similarity to all or part of SEQ ID NO:1 or SEQ ID NO:2 or SEQ ID NO:3 or SEQ ID NO:4 or SEQ ID NO:5 or SEQ ID NO:6 and/or is capable of hybridizing to SEQ ID NO:1 or SEQ ID NO:2 or SEQ ID NO:3 or SEQ ID NO:4 or SEQ ID NO:5 or SEQ ID NO:6 or a complementary form thereof under low stringency conditions at 42° C.

Reference to “homolog” includes other mammalian homologs such as from a human.

The preferred genetic sequence of the present invention are referred to herein as CXS-740 (SEQ ID NO:1), CXS-741 (SEQ ID NO:2), CXS-742 (SEQ ID NO:3), CXS-743 (SEQ ID NO:4), CXS-744 (SEQ ID NO:5) and CXS-745 (SEQ ID NO:6). The expression products encoded by CXS-740, CXS-741, CXS-742, CXS-743, CXS-744 and CXS-745 are referred to herein as CXS-740, CXS-741, CXS-742, CXS-743, CXS-744 and CXS-745, respectively (i.e. non-itallized). The expression product may be an RNA (e.g. mRNA) or a protein. Where the expression product is an RNA, the present invention extends to RNA-related molecules associated thereto such as RNAi or intron or exon sequences therefrom or short, interfering RNA (si-RNA) or complexes comprising same. Most preferred molecules are CXS-741 (SEQ ID NO:2) and CXS-744 (SEQ ID NO:5).

Even yet another aspect of the present invention relates to a composition comprising CXS-740, CXS-741, CXS-742, CXS-743, CXS-744 and/or CXS-745 or their derivatives, homologs, analogs or mimetics or agonists or antagonists of CXS-740, CXS-741, CXS-742, CXS-743, CXS-744 and/or CXS-745 together with one or more pharmaceutically acceptable carriers and/or diluents.

The present invention is particularly directed to human homologs and orthologs of the genes identified in P. obesus and their use in therapy and diagnosis.

Another aspect of the present invention contemplates, therefore, a method for treating a subject comprising administering to said subject a treatment effective amount of CXS-740, CXS-741, CXS-742, CXS-743, CXS-744 and/or CXS-745 or a derivative, homolog, analog or mimetic thereof or a genetic sequence encoding same or an agonist or antagonist of CXS-740, CXS-741, CXS-742, CXS-743, CXS-744 and/or CXS-745 activity or of CXS-740, CXS-741, CXS-742, CXS-743, CXS-744 and/or CXS-745 gene expression for a time and under conditions sufficient to effect treatment.

In accordance with this and other aspects of the present invention, treatments contemplated herein include but are not limited to metabolic disorders such as diabetes (Types I or II), or a complication therefrom, inflammation, mitochondrial dysfunction, myopathies, genetic disorders, cancers, energy imbalance and/or obesity as well as modulating apoptosis, signal transduction and/or nuclear targeting. Treatment may be by the administration of a pharmaceutical composition or genetic sequences via gene therapy, antisense therapy or sense or RNAi- or si-RNA-mediated therapy. Treatment is contemplated for human subjects as well as animals such as animals important to livestock industry.

Another aspect of the present invention contemplates a method of treating a subject suffering from diabetes or a complication thereof, said method comprising administering to said subject an effective amount of an agent for a time and under conditions sufficient to down-regulate the level or activity CXS-741 (SEQ ID NO:2) and/or CXS-744 (SEQ ID NO:5).

Still another aspect of the present invention provides a method of treating a subject suffering from an inflammatory condition said method comprising administering to said subject an effective amount of an agent for a time and under conditions sufficient to down-regulate the level of activity of CXS-741 (SEQ ID NO:2) and/or CXS-744 (SEQ ID NO:5).

A further aspect of the present invention is directed to a diagnostic agent for use in monitoring or diagnosing conditions such as but not limited to diabetes, mitochondrial dysfunction, genetic disorders, cancers, energy imbalance and/or obesity as well as monitoring apoptosis, signal transduction and/or nuclear targeting, said diagnostic agent selected from an antibody to one or more of CXS-740, CXS-741, CXS-742, CXS-743, CXS-744 and/or CXS-745 or its derivatives, homologs, analogs or mimetics and a genetic sequence comprising or capable of annealing to a nucleotide strand associated with CXS-740, CXS-741, CXS-742, CXS-743, CXS-744 and/or CXS-745 useful inter alia in PCR, hybridization, RFLP analysis or AFLP analysis.

Yet another aspect of the present invention is directed to the use of CXS-741 and/or CXS-744 or CXS-741 and/or CXS-744 or a homolog thereof in the manufacture of a medicament for the treatment of diabetes, a complication of diabetes and/or inflammation.

TABLE 1
Summary of CXS Genes

| Gene | SEQ ID NO: | Method of isolation | Expression |
|---|---|---|---|
| CXS-740 | 1 | Sequence Trap | Sushi domain containing |
| | | | 2 (Susd2) gene |
| CXS-741 | 2 | Sequence Trap | Decorin (Dcn) gene |
| CXS-742 | 3 | Sequence Trap | Periostin gene |
| CXS-743 | 4 | Sequence Trap | Collagen and calcium |
| | | | binding EGF domain 1 |
| | | | (CCBE1) gene |
| CXS-744 | 5 | Sequence Trap | Thioredoxin-like protein |
| | | | p19 (TLP19) gene |
| CXS-745 | 6 | Sequence Trap | MAPK activity protein |
| | | | PM20, PM21 gene |

A summary of sequence identifiers used throughout the subject specification is provided in Table 2.

TABLE 2
Summary of Sequence Identifiers

| SEQUENCE | |
|---|---|
| ID NO: | DESCRIPTION |
| 1 | Partial Nucleotide sequence of CXS-740 from P. obesus |
| 2 | Partial Nucleotide sequence of CXS-741 from P. obesus |
| 3 | Partial Nucleotide sequence of CXS-742 from P. obesus |
| 4 | Partial Nucleotide sequence of CXS-743 from P. obesus |
| 5 | Partial Nucleotide sequence of CXS-744 from P. obesus |
| 6 | Partial Nucleotide sequence of CXS-745 from P. obesus |
| 7 | Primer with Not1 restriction site (synthetic DNA) |
| 8 | Forward PCR primer (synthetic DNA) |
| 9 | Reverse PCR primer (synthetic DNA) |
| 10 | Forward PCR primer (synthetic DNA) |
| 11 | Reverse PCR primer (synthetic DNA) |

### BRIEF DESCRIPTION OF THE DRAWINGS

FIG. 1 is a representation of the nucleotide sequence of CXS-740 from P. obesus.

FIG. 2 is a representation of the nucleotide sequence of CXS-741 from P. obesus.

FIG. 3 is a representation of the nucleotide sequence of CXS-742 from P. obesus.

FIG. 4 is a representation of the nucleotide sequence of CXS-743 from P. obesus.

FIG. 5 is a representation of the nucleotide sequence of CXS-744 from P. obesus.

FIG. 6 is a representation of the nucleotide sequence of CXS-745 from P. obesus.

### DETAILED DESCRIPTION OF THE INVENTION

Prior to describing embodiments of the present invention in detail, it is to be understood that unless otherwise indicated, the subject invention is not limited to specific therapeutic components, manufacturing methods, dosage regimens, or the like, as such may vary. It is also to be understood that the terminology used herein is for the purpose of describing particular embodiments only and is not intended to be limiting.

It must also be noted that, as used in the subject specification, the singular forms “a”, “an” and “the” include plural aspects unless the context clearly dictates otherwise. Thus, for example, reference to “an agent” includes a single agent, as well as two or more agents; reference to “a gene” includes a single gene, as well as two or more genes; and so forth.

Reference herein to an “agent” should be understood as a reference to any proteinaceous or non-proteinaceous molecule derived from natural, recombinant or synthetic sources. Useful sources include the screening of naturally produced libraries, chemical molecule libraries as well as combinatorial libraries, phage display libraries and in vitro translation-based libraries. Particularly useful agents are those identified by the Signal Sequence Trap method. The agents may, however, be any proteinaceous molecules such as peptides, polypeptides and proteins or non-proteinaceous molecules such as nucleic acid molecules and small to large natural or synthetically derived organic and inorganic molecules and include antagonists and agonists of the proteins identified by the Signal Sequence Trap (SST) method. The agents may, therefore, also be immunoglobulins such as antibodies or fragments or synthetic or modified forms thereof.

The terms “agent”, “compound”, “active agent”, “pharmacologically active agent”, “medicament”, “active” and “drug” may be used interchangeably herein to refer to any agent that induces a desired pharmacological and/or physiological effect. The terms also encompass pharmaceutically acceptable and pharmacologically active ingredients of those active agents specifically mentioned herein including but not limited to salts, esters, amides, prodrugs, active metabolites, analogs and the like. When the terms “agent”, “compound”, “active agent”, “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc.

The “Signal Sequence Trap” method used in accordance with the present invention refers to a procedure whereby a cDNA library is generated, inserted 5′ upstream of a genetic sequence encoding a cytokine. The cDNA library is then transfected into a cell line dependent on the cytokine exported from the cell for survival. Only cells in which a functional signal sequence facilitates export of the cytokine from the cell will survive.

A “sample” includes a biological fluid sample such as but not limited to whole blood, blood plasma, serum, mucus, urine, semen, respiratory fluid, lymph fluid, saliva and other tissue secretions or fluid. Preferred fluid is whole blood, blood plasma and serum.

Accordingly, one aspect of the present invention contemplates a method for identifying a signal sequence which facilitates export of a cytokine out of a cell, said method comprising generating a cDNA library and inserting DNA fragments in a vector upstream of a genetic sequence encoding a cytokine such that upon expression, the inserted DNA fragment encodes a molecule operably fused to said cytokine, transfecting a cell line dependent on said cytokine for survival and screening for live cells wherein live cells is indicative of a DNA fragment encoding a signal sequence.

Preferably, the cytokine is IL-3. However, any of a range of cytokines may be employed. In addition, non-cytokine molecules may also be employed.

Once a clone containing a functional signal sequence is identified, BLAST or microarray analysis is done to identify a gene which naturally comprises the signal sequence-encoding nucleotide sequence.

The present invention is predicated in part on the identification of genes associated inter alia with diabetes, complications associated with diabetes, such as blindness, nephropathy and/or cardiovascular disease, inflammation, mitochondrial dysfunction, a myopathy, a genetic disorder or a cancer or in modulating apoptosis, signal transduction and/or nuclear targeting. The genes were identified using the Signal Sequence Trap and microarray methods described herein.

Preferred conditions include diabetes (Type I and Type II) and inflammation. Reference to “inflammation” includes acne, angina, arthritis, aspiration pneumonia, disease, empyema, gastroenteritis, inflammation, intestinal flu, NEC, necrotizing enterocolitis, pelvic inflammatory disease, pharyngitis, PID, pleurisy, raw throat, redness, rubor, sore throat, stomach flu, urinary tract infection, Chronic Inflammatory Demyelinating Polyneuropathy and Chronic Inflammatory Demyelinating Polyradiculoneuropathy.

Accordingly, another aspect of the present invention provides a nucleic acid molecule comprising a sequence of nucleotides encoding or complementary to a sequence encoding an expression product or a derivative, or fragment or homolog, analog or mimetic or portion thereof wherein said nucleic acid molecule is associated with one or more of mitochondrial dysfunction, myopathies, genetic disorders or cancer or in modulating apoptosis, signal transduction and/or nuclear targeting and is identified as being a secreted or cell surface molecule and involved in metabolic signalling in a cell or group of cells.

The expression product may be a peptide, polypeptide or protein or mRNA or may be an exon or intron spliced, for example, from an RNA construct. The expression product may also be a hairpin structure which induces or is associated with RNAi.

As used herein a fragment includes a part, portion, region, domain, N-terminal fragment, a C-terminal fragment, an internal fragments and/or an enzymatically cleaved protein, such as by a membrane cleaving protease.

More particularly, the present invention provides a nucleic acid molecule composition. Therefore, the present invention provides compositions which comprise one or more of a nucleic acid molecule selected from the list consisting of SEQ ID NO:1 (CXS-740), SEQ ID NO:2 (CXS-741), SEQ ID NO:3 (CXS-742), SEQ ID NO:4 (CXS-743), SEQ ID NO:5 (CXS-744) and SEQ ID NO:6 (CXS-745). The corresponding expression products are referred to as CXS-740, CXS-741, CXS-742, CXS-743, CXS-744 and CXS-745.

Hence, the present invention contemplates a method for identifying a nucleic acid molecule comprising a signal sequence which facilitates export of a cytokine out of a cell, said method comprising generating a cDNA library and inserting DNA fragments into a vector upstream of a genetic sequence encoding a cytokine such that upon expression, the inserted DNA fragment encodes a molecule operably fused to said cytokine, transfecting a cell line dependent on said cytokine for survival and screening for live cells wherein live cells is indicative of a DNA fragment encoding a signal sequence wherein DNA fragments which enable transport of the cytokine comprise a nucleotide sequence selected from the list consisting of:
(i) a nucleotide sequence as set forth in SEQ ID NO:2 (CXS-741) or a nucleotide sequence having at least about 90% identity thereto or a nucleotide sequence capable of hybridizing to SEQ ID NO:2 (CXS-741) or its complementary form; and
(ii) a nucleotide sequence as set forth in SEQ ID NO:5 (CXS-744) or a nucleotide sequence having at least about 90% identity thereto or a nucleotide sequence capable of hybridizing to SEQ ID NO:5 (CXS-744) or its complementary form.

Another aspect of the present invention provides a method for assessing the presence or absence of diabetes, complications associated with diabetes, such as blindness, nephropathy and/or cardiovascular disease, inflammation, mitochondrial dysfunction, myopathy, genetic disorder or cancer or in modulating apoptosis, signal transduction and/or nuclear targeting by determining the level of expression of a nucleic acid molecule comprising a nucleotide sequence encoding or complementary to a sequence encoding an expression product or a derivative, homolog or mimetic thereof wherein said nucleotide sequence is as substantially set forth in SEQ ID NO:1 (CXS-740) or SEQ ID NO:2 (CXS-741) or SEQ ID NO:3 (CXS-742) or SEQ ID NO:4 (CXS-743) or SEQ ID NO:5 (CXS-744) or SEQ ID NO:6 (CXS-745) or a nucleotide sequence having at least about 30% similarity to all or part of SEQ ID NO:1 or SEQ ID NO:2 or SEQ ID NO:3 or SEQ ID NO:4 or SEQ ID NO:5 or SEQ ID NO:6 and/or is capable of hybridizing to one or more of SEQ ID NO:1 or SEQ ID NO:2 or SEQ ID NO:3 or SEQ ID NO:4 or SEQ ID NO:5 or SEQ ID NO:6 or their complementary forms under low stringency conditions at a specified temperature and wherein elevated or reduced levels of expression of one or more of these sequences is indicative of one or more of diabetes or complications thereof, inflammation, mitochondrial dysfunction, myopathy, genetic disorder or cancer or in modulating apoptosis, signal transduction and/or nuclear targeting.

Reference herein to similarity or identity is generally at a level of comparison of at least 15 consecutive or substantially consecutive nucleotides (or corresponding amino acids) such as at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399 or 400 consecutive or substantially consecutive nucleotides (or amino acids). Preferred percentage similarities or identities have at least about 40%, at least about 50%, at least about 60%, at least about 70%, at least about 80% and at least about 90% or above. Examples include 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100%.

The term “similarity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length, examples include 12, 13, 14, 15, 16, 17, 18, 19, 20, 21, 22, 23, 24 and 25. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as for example disclosed by Altschul et al. (Nucl. Acids Res. 25: 3389, 1997). A detailed discussion of sequence analysis can be found in Unit 19.3 of Ausubel et al. (“Current Protocols in Molecular Biology” John Wiley & Sons Inc, 1994-1998, Chapter 15).

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C., such as 25, 26, 27, 28, 29, 30, 31, 32, 33, 34, 35, 36, 37, 38, 39, 40, 41 and 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide, such as 16, 17, 18, 19, 20, 21, 22, 23, 24, 25, 26, 27, 28, 29 and 30% and from at least about 0.5 M to at least about 0.9 M salt, such as 0.5, 0.6, 0.7, 0.8 and 0.9 M for hybridization, and at least about 0.5 M to at least about 0.9 M salt, such as 0.5, 0.6, 0.7, 0.8 and 0.9 M for washing conditions, or high stringency, which includes and encompasses from at least about 31% v/v to at least about 50% v/v formamide, such as 31, 32, 33, 34, 35, 36, 37, 38, 39, 40, 41, 42, 43, 44, 45, 46, 47, 48, 49 and 50% v/v formamide and from at least about 0.01 M to at least about 0.15 M salt, such as 0.01, 0.02, 0.03, 0.04, 0.05, 0.06, 0.07, 0.08, 0.09, 0.10, 0.11, 0.12, 0.13, 0.14 and 0.15 M for hybridization, and at least about 0.01 M to at least about 0.15 M salt, such as 0.01, 0.02, 0.03, 0.04, 0.05, 0.06, 0.07, 0.08, 0.09, 0.10, 0.11, 0.12, 0.13, 0.14 and 0.15 M for washing conditions. In general, washing is carried out Tₘ=69.3+0.41 (G+C) % (Marmur and Doty, J. Mol. Biol. 5: 109, 1962). However, the Tₘ of a duplex DNA decreases by 1° C. with every increase of 1% in the number of mismatch base pairs (Bonner and Laskey, Eur. J. Biochem. 46: 83, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

In a preferred embodiment, the present invention provides A method for the prognosis of diabetes or a complication arising from diabetes in a subject, said method comprising screening for elevated levels of CXS-741 (SEQ ID NO:2) and/or CXS-744 (SEQ ID NO:5) protein or mRNA encoding said protein or a homolog thereof in a biological sample from said subject wherein an elevated level is indicative of diabetes or a complication arising therefrom or a likelihood of development of same.

In another embodiment, the present invention contemplates A method for the prognosis of an inflammatory condition in a subject, said method comprising screening for elevated levels of CXS-741 (SEQ ID NO:2) and/or CXS-744 (SEQ ID NO:5) protein or mRNA encoding said protein or a homolog thereof in a biological sample from said subject wherein an elevated level is indicative of an inflammatory condition.

Another aspect of the present invention is directed to an isolated nucleic acid molecule comprising a sequence of nucleotides said nucleic acid molecule differentially expressed in cells from a subject having one or more diseases and/or conditions wherein the nucleic acid molecule is selected from the list consisting of:
(i) a nucleic acid molecule comprises a nucleotide sequence as set forth in SEQ ID NO:1 (CXS-740) or a nucleotide sequence having at least about 50% identity thereto or a nucleotide sequence capable of hybridizing to SEQ ID NO:1 (CXS-740) or its complementary form;
(ii) a nucleic acid molecule comprises a nucleotide sequence as set forth in SEQ ID NO:2 (CXS-741) or a nucleotide sequence having at least about 50% identity thereto or a nucleotide sequence capable of hybridizing to SEQ ID NO:2 (CXS-741) or its complementary form;
(iii) a nucleic acid molecule comprises a nucleotide sequence as set forth in SEQ ID NO:3 (CXS-742) or a nucleotide sequence having at least about 50% identity thereto or a nucleotide sequence capable of hybridizing to SEQ ID NO:3 (CXS-742) or its complementary form;
(iv) a nucleic acid molecule comprises a nucleotide sequence as set forth in SEQ ID NO:4 (CXS-743) or a nucleotide sequence having at least about 50% identity thereto or a nucleotide sequence capable of hybridizing to SEQ ID NO:4 (CXS-743) or its complementary form;
(v) a nucleic acid molecule comprises a nucleotide sequence as set forth in SEQ ID NO:5 (CXS-744) or a nucleotide sequence having at least about 55% identity thereto or a nucleotide sequence capable of hybridizing to SEQ ID NO:5 (CXS-744) or its complementary form;
(vi) a nucleic acid molecule comprises a nucleotide sequence as set forth in SEQ ID NO:6 (CXS-745) or a nucleotide sequence having at least about 55% identity thereto or a nucleotide sequence capable of hybridizing to SEQ ID NO:6 (CXS-745) or its complementary form

A further aspect of the present invention provides a nucleic acid molecule or derivative, homolog or analog thereof associated with one or more of diabetes, complications associated with diabetes, such as blindness, nephropathy and/or cardiovascular disease, inflammation, mitochondrial dysfunction, myopathy, a genetic disorder or cancer or in modulating apoptosis, signal transduction and/or nuclear targeting said nucleic acid molecule comprising a nucleotide sequence encoding, or a nucleotide sequence complementary to a sequence encoding an expression product wherein said nucleotide sequence is substantially as set forth in SEQ ID NO:1 or a derivative, homolog or mimetic thereof or having at least about 50% identity to all or part of SEQ ID NO:1.

Yet another aspect of the present invention provides a nucleic acid molecule or derivative, homolog or analog thereof associated with one or more of diabetes, complications associated with diabetes, such as blindness, nephropathy and/or cardiovascular disease, inflammation, mitochondrial dysfunction, myopathy, a genetic disorder or cancer or in modulating apoptosis, signal transduction and/or nuclear targeting said nucleic acid molecule comprising a nucleotide sequence encoding, or a nucleotide sequence complementary to a sequence encoding an expression product wherein said nucleotide sequence is substantially as set forth in SEQ ID NO:2 or a derivative, homolog or mimetic thereof or having at least about 50% identity to all or part of SEQ ID NO:2.

Still yet another aspect of the present invention provides a nucleic acid molecule or derivative, homolog or analog thereof associated with one or more of diabetes, complications associated with diabetes, such as blindness, nephropathy and/or cardiovascular disease, inflammation, mitochondrial dysfunction, myopathy, a genetic disorder or cancer or in modulating apoptosis, signal transduction and/or nuclear targeting said nucleic acid molecule comprising a nucleotide sequence encoding, or a nucleotide sequence complementary to a sequence encoding an expression product wherein said nucleotide sequence is substantially as set forth in SEQ ID NO:3 or a derivative, homolog or mimetic thereof or having at least about 50% identity to all or part of SEQ ID NO:3.

Another aspect of the present invention provides a nucleic acid molecule or derivative, homolog or analog thereof associated with one or more of diabetes, complications associated with diabetes, such as blindness, nephropathy and/or cardiovascular disease, inflammation, mitochondrial dysfunction, myopathy, a genetic disorder or cancer or in modulating apoptosis, signal transduction and/or nuclear targeting said nucleic acid molecule comprising a nucleotide sequence encoding, or a nucleotide sequence complementary to a sequence encoding an expression product wherein said nucleotide sequence is substantially as set forth in SEQ ID NO:4 or a derivative, homolog or mimetic thereof or having at least about 50% identity to all or part of SEQ ID NO:4.

Another aspect of the present invention provides a nucleic acid molecule or derivative, homolog or analog thereof associated with one or more of diabetes, complications associated with diabetes, such as blindness, nephropathy and/or cardiovascular disease, inflammation, mitochondrial dysfunction, myopathy, a genetic disorder or cancer or in modulating apoptosis, signal transduction and/or nuclear targeting said nucleic acid molecule comprising a nucleotide sequence encoding, or a nucleotide sequence complementary to a sequence encoding an expression product wherein said nucleotide sequence is substantially as set forth in SEQ ID NO:5 or a derivative, homolog or mimetic thereof or having at least about 50% identity to all or part of SEQ ID NO:5.

Another aspect of the present invention provides a nucleic acid molecule or derivative, homolog or analog thereof associated with one or more of diabetes, complications associated with diabetes, such as blindness, nephropathy and/or cardiovascular disease, inflammation, mitochondrial dysfunction, myopathy, a genetic disorder or cancer or in modulating apoptosis, signal transduction and/or nuclear targeting said nucleic acid molecule comprising a nucleotide sequence encoding, or a nucleotide sequence complementary to a sequence encoding an expression product wherein said nucleotide sequence is substantially as set forth in SEQ ID NO:6 or a derivative, homolog or mimetic thereof or having at least about 50% identity to all or part of SEQ ID NO:6.

The expression pattern of CXS-740, CXS-741, CXS-742, CXS-743, CXS-744 and CXS-745 has been determined, inter alia, to indicate an involvement in the regulation of one or more processes associated with one or more of diabetes, complications associated with diabetes, such as blindness, nephropathy and/or cardiovascular disease, inflammation, mitochondrial dysfunction, myopathy, a genetic disorder or cancer or in modulating apoptosis, signal transduction and/or nuclear targeting said nucleic acid molecule. The subject nucleic acid molecules are preferably a sequence of deoxyribonucleic acids such as a cDNA sequence or a genomic sequence. A genomic sequence may also comprise exons and introns. A genomic sequence may also include a promoter region or other regulatory regions. The present invention extends, however, to expression products such as mRNA, introns and exons which may also be involved in genetic networking, whether or not they are translated into proteins. Furthermore, the expression products may include complexes comprising RNA or may comprising RNAi or RNAi-type molecules.

A homolog is considered to be a CXS-740, CXS-741, CXS-742, CXS-743, CXS-744 and CXS-745 gene from another animal species. The present invention extends to the homologous gene, as determined by nucleotide sequence and/or amino acid sequences and/or function, from primates, including humans, marmosets, orangutans and gorillas, livestock animals (e.g. cows, sheep, pigs, horses, donkeys), laboratory test animals (e.g. mice, rats, guinea pigs, hamsters, rabbits), companion animals (e.g. cats, dogs) and captured wild animals (e.g. rodents, foxes, deer, kangaroos). The present invention also contemplates deimmunized forms of the expression products from one species relative to another species. In one preferred embodiment, the deimmunized form of the expression product is a mamalianized form relative to a particular target animal. In a most preferred embodiment where the target mammal is a human, the present invention contemplates use of a humanized form of a non-human expression product.

CXS-740, CXS-741, CXS-742, CXS-743, CXS-744 and CXS-745 and their derivatives and homologs may be in isolated or purified form and/or may be ligated to a vector such as an expression vector. Expression may be in a eukaryotic cell line (e.g. mammalian, insect or yeast cells) or in microbial cells (e.g. E. coli) or both.

By “isolated” is meant a nucleic acid molecule having undergone at least one purification step and this is conveniently defined, for example, by a composition comprising at least about 10% subject nucleic acid molecule, preferably at least about 20%, more preferably at least about 30%, still more preferably at least about 40-50%, even still more preferably at least about 60-70%, yet even still more preferably 80-90% or greater of subject nucleic acid molecule relative to other components as determined by molecular weight, encoding activity, nucleotide sequence, base composition or other convenient means. The nucleic acid molecule of the present invention may also be considered, in a preferred embodiment, to be biologically pure. The nucleic acid molecule may be ligated to an expression vector capable of expression in a prokaryotic cell (e.g. E. coli) or a eukaryotic cell (e.g. yeast cells, fungal cells, insect cells, mammalian cells or plant cells). The nucleic acid molecule may be ligated or fused or otherwise associated with a nucleic acid molecule encoding another entity such as, for example, a signal peptide. It may also comprise additional nucleotide sequence information fused, linked or otherwise associated with it either at the 3′ or 5′ terminal portions or at both the 3′ and 5′ terminal portions. The nucleic acid molecule may also be part of a vector, such as an expression vector.

In a particularly preferred embodiment, the nucleotide sequence corresponding to CXS-740 is a cDNA sequence comprising a sequence of nucleotides as set forth in SEQ ID NO:1 or a derivative, homolog or analog thereof including a nucleotide sequence having similarity to SEQ ID NO:1.

In another particularly preferred embodiment, the nucleotide sequence corresponding to CXS-741 is a cDNA sequence comprising a sequence of nucleotides as set forth in SEQ ID NO:2 or a derivative, homolog or analog thereof including a nucleotide sequence having similarity to SEQ ID NO:2.

In still another particularly preferred embodiment, the nucleotide sequence corresponding to CXS-742 is a cDNA sequence comprising a sequence of nucleotides as set forth in SEQ ID NO:3 or a derivative, homolog or analog thereof including a nucleotide sequence having similarity to SEQ ID NO:3.

In a further particularly preferred embodiment, the nucleotide sequence corresponding to CXS-743 is a cDNA sequence comprising a sequence of nucleotides as set forth in SEQ ID NO:4 or a derivative, homolog or analog thereof including a nucleotide sequence having similarity to SEQ ID NO:4.

In still a further particularly preferred embodiment, the nucleotide sequence corresponding to CXS-744 is a cDNA sequence comprising a sequence of nucleotides as set forth in SEQ ID NO:5 or a derivative, homolog or analog thereof including a nucleotide sequence having similarity to SEQ ID NO:5.

Yet another preferred embodiment, the nucleotide sequence corresponding to CXS-745 is a cDNA sequence comprising a sequence of nucleotides as set forth in SEQ ID NO:6 or a derivative, homolog or analog thereof including a nucleotide sequence having similarity to SEQ ID NO:6.

The nucleic acid molecule may be ligated to an expression vector capable of expression in a prokaryotic cell (e.g. E. coli) or a eukaryotic cell (e.g. yeast cells, fungal cells, insect cells, mammalian cells or plant cells). The nucleic acid molecule may be ligated or fused or otherwise associated with a nucleic acid molecule encoding another entity such as, for example, a signal peptide. It may also comprise additional nucleotide sequence information fused, linked or otherwise associated with it either at the 3′ or 5′ terminal portions or at both the 3′ and 5′ terminal portions. The nucleic acid molecule may also be part of a vector, such as an expression vector.

The derivatives of the nucleic acid molecule of the present invention include oligonucleotides, PCR primers, antisense molecules, molecules suitable for use in co-suppression (e.g. RNAi) and fusion nucleic acid molecules. Ribozymes and DNA enzymes are also contemplated by the present invention directed to CXS-740, CXS-741, CXS-742, CXS-743, CXS-744 and CXS-745 or their mRNA. Derivatives and homologs of CXS-740, CXS-741, CXS-742, CXS-743, CXS-744 and CXS-745 are conveniently encompassed by those nucleotide sequences capable of hybridizing to SEQ ID NO:1, SEQ ID NO:2 or SEQ ID NO:3 or SEQ ID NO:4 or SEQ ID NO:5 or SEQ ID NO:6 or their complementary form under low stringency conditions.

The present invention extends to expression products of CXS-740, CXS-741, CXS-742, CXS-743, CXS-744 and CXS-745. The preferred expression products are proteins or mutants, derivatives, homologs or analogs thereof as well as a range of RNA molecules.

An expression product includes an RNA molecule such as a mRNA transcript as well as a protein. Some genes are non-protein encoding genes and produce mRNA or other RNA type molecules and are involved in regulation by RNA:DNA, RNA:RNA or RNA:protein interaction. The RNA (e.g. mRNA) may act directly or via the induction of other molecules such as RNAi or via products mediated from splicing events (e.g. exons or introns). Other genes encode mRNA transcripts which are then translated into proteins. A protein includes a polypeptide. The differentially expressed nucleic acid molecules, therefore, may encode mRNAs only or, in addition, proteins. Both mRNAs and proteins are forms of “expression products”.

Derivatives include fragments, parts, portions, mutants, variants and mimetics from natural, synthetic or recombinant sources including fusion proteins. Parts or fragments include, for example, active regions of CXS-740, CXS-741, CXS-742, CXS-743, CXS-744 and CXS-745. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences include fusions with other peptides, polypeptides or proteins.

Chemical and functional equivalents of CXS-740, CXS-741, CXS-742, CXS-743, CXS-744 and CXS-745 should be understood as molecules exhibiting any one or more of the functional activities of these molecules and may be derived from any source such as being chemically synthesized or identified via screening processes such as natural product screening.

The derivatives include fragments having particular epitopes or parts of the entire protein fused to peptides, polypeptides or other proteinaceous or non-proteinaceous molecules.

Another aspect of the present invention provides an isolated protein or other expression product or a derivative, homolog, analog or mimetic thereof which is associated with one or more of diabetes, complications associated with diabetes, such as blindness, nephropathy and/or cardiovascular disease, inflammation, mitochondrial disease, myopathy, a genetic disorder or cancer or in modulating apoptosis, signal transduction and/or nuclear targeting.

In a preferred aspect of the present invention, there is provided an isolated protein or derivative, homolog, analog, fragment or mimetic thereof wherein said protein or polypeptide comprises an amino acid sequence encoded by SEQ ID NO:1 (CXS-740) or an amino acid sequence having at least 30% similarity to all or part thereof and wherein said protein or expression product is associated with one or more of diabetes, complications associated with diabetes, such as blindness, nephropathy and/or cardiovascular disease, inflammation, mitochondrial dysfunction, myopathy, a genetic disorder or cancer or in modulating apoptosis, signal transduction and/or nuclear targeting.

In another preferred aspect of the present invention, there is provided an isolated protein or derivative, homolog, analog, fragment or mimetic thereof wherein said protein or polypeptide comprises an amino acid sequence encoded by SEQ ID NO:2 (CXS-741) or an amino acid sequence having at least 50% similarity to all or part thereof and wherein said protein or expression product is associated with one or more of diabetes, complications associated with diabetes, such as blindness, nephropathy and/or cardiovascular disease, inflammation, mitochondrial dysfunction, myopathy, a genetic disorder or cancer or in modulating apoptosis, signal transduction, nuclear targeting and/or inflammation.

As indicated above, the term “inflammatory diseases and disorders” encompasses those disease and disorders which result in one or more inflammatory response symptoms such as redness, swelling, pain, and a feeling of heat in certain areas. Inflammatory pain is often associated with the following diseases acne, angina, arthritis, aspiration pneumonia, disease, empyema, gastroenteritis, inflammation, intestinal flu, NEC, necrotizing enterocolitis, pelvic inflammatory disease, pharyngitis, PID, pleurisy, raw throat, redness, rubor, sore throat, stomach flu and urinary tract infections, Chronic Inflammatory Demyelinating Polyneuropathy and Chronic Inflammatory Demyelinating Polyradiculoneuropathy. Accordingly, the compositions and methods of the present invention ameliorate or decrease or prevent or treat the inflammatory processes.

In still another preferred aspect of the present invention, there is provided an isolated protein or derivative, homolog, analog, fragment or mimetic thereof wherein said protein or polypeptide comprises an amino acid sequence encoded by SEQ ID NO:3 (CXS-742) or an amino acid sequence having at least 50% similarity to all or part thereof and wherein said protein or expression product is associated with one or more of diabetes, complications associated with diabetes, such as blindness, nephropathy and/or cardiovascular disease, inflammation, mitochondrial dysfunction, myopathy, a genetic disorder or cancer or in modulating apoptosis, signal transduction and/or nuclear targeting.

In a further preferred aspect of the present invention, there is provided an isolated protein or derivative, homolog, analog, fragment or mimetic thereof wherein said protein or polypeptide comprises an amino acid sequence encoded by SEQ ID NO:4 (CXS-743) or an amino acid sequence having at least 50% similarity to all or part thereof and wherein said protein or expression product is associated with one or more of diabetes, complications associated with diabetes, such as blindness, nephropathy and/or cardiovascular disease, inflammation, mitochondrial dysfunction, myopathy, a genetic disorder or cancer or in modulating apoptosis, signal transduction and/or nuclear targeting.

In still a further preferred aspect of the present invention, there is provided an isolated protein or derivative, homolog, analog, fragment or mimetic thereof wherein said protein or polypeptide comprises an amino acid sequence encoded by SEQ ID NO:5 (CXS-744) or an amino acid sequence having at least 50% similarity to all or part thereof and wherein said protein or expression product is associated with one or more of diabetes, complications associated with diabetes, such as blindness, nephropathy and/or cardiovascular disease, inflammation, mitochondrial dysfunction, myopathy, a genetic disorder or cancer or in modulating apoptosis, signal transduction and/or nuclear targeting.

Yet another preferred aspect of the present invention, there is provided an isolated protein or derivative, homolog, analog, fragment or mimetic thereof wherein said protein or polypeptide comprises an amino acid sequence encoded by SEQ ID NO:6 (CXS-745) or an amino acid sequence having at least 50% similarity to all or part thereof and wherein said protein or expression product is associated with one or more of diabetes, complications associated with diabetes, such as blindness, nephropathy and/or cardiovascular disease, inflammation, mitochondrial dysfunction, myopathy, a genetic disorder or cancer or in modulating apoptosis, signal transduction and/or nuclear targeting.

Reference herein to CXS-740, CXS-741, CXS-742, CXS-743, CXS-744 and CXS-745 includes reference to isolated or purified naturally occurring CXS-740, CXS-741, CXS-742, CXS-743, CXS-744 and CXS-745 protein or expression product molecules as well as any derivatives, homologs, analogs and mimetics thereof. Derivatives include parts, fragments and portions of CXS-740, CXS-741, CXS-742, CXS-743, CXS-744 and CXS-745 as well as single and multiple amino acid substitutions, deletions and/or additions to CXS-740, CXS-741, CXS-742, CXS-743, CXS-744 and CXS-745. A derivative of CXS-740, CXS-741, CXS-742, CXS-743, CXS-744 and CXS-745 is conveniently encompassed by molecules encoded by a nucleotide sequence capable of hybridizing to SEQ ID NO:1, SEQ ID NO:2, SEQ ID NO:3, SEQ ID NO:4 or SEQ ID NO:5 or SEQ ID NO:6, respectively under low stringency conditions at a specified temperature.

Other derivatives of CXS-740, CXS-741, CXS-742, CXS-743, CXS-744 and CXS-745 include chemical analogs. Analogs of ACXS-740, CXS-741, CXS-742, CXS-743, CXS-744 and CXS-745 contemplated herein include, but are not limited to, modifications to side chains, incorporation of unnatural amino acids and/or their derivatives during peptide, polypeptide or protein synthesis and the use of crosslinkers and other methods which impose confirmational constraints on the proteinaceous molecule or their analogs.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 3.

TABLE 3
Codes for non-conventional amino acids

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-Nmethylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylasparatate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethy)glycine | Nthr |
| D-N-methylhistidine | Dnmhis | N-(hydroxyethy))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogs by introducing covalent bonds such as forming an amide bond between the N and C termini, between two side chains or between a side chain and the N or C terminus.

All such modifications may also be useful in stabilizing the CXS-740, CXS-741, CXS-742, CXS-743, CXS-744 and CXS-745 molecule for use in in vivo administration protocols or for diagnostic purposes.

As stated above, the expression product may be an RNA or protein.

The term “protein” should be understood to encompass peptides, polypeptides and proteins. The protein may be glycosylated or unglycosylated and/or may contain a range of other molecules fused, linked, bound or otherwise associated to the protein such as amino acids, lipids, carbohydrates or other peptides, polypeptides or proteins. Reference hereinafter to a “protein” includes a protein comprising a sequence of amino acids as well as a protein associated with other molecules such as amino acids, lipids, carbohydrates or other peptides, polypeptides or proteins.

In a particularly preferred embodiment, the expression product is encoded by a sequence of nucleotides as set forth in SEQ ID NO:1 or a derivative, homolog or analog thereof including a nucleotide sequence having at least about 50% identity to SEQ ID NO:1.

In another particularly preferred embodiment, the expression product is encoded by a sequence of nucleotides as set forth in SEQ ID NO:2 or a derivative, homolog or analog thereof including a nucleotide sequence having at least about 50% identity to SEQ ID NO:2.

In still another particularly preferred embodiment, the expression product is encoded by a sequence of nucleotides as set forth in SEQ ID NO:3 or a derivative homolog or analog thereof including a nucleotide sequence having at least about 50% identity to SEQ ID NO:3.

In still another particularly preferred embodiment, the expression product is encoded by a sequence of nucleotides as set forth in SEQ ID NO:4 or a derivative homolog or analog thereof including a nucleotide sequence having at least about 50% identity to SEQ ID NO:4.

In still another particularly preferred embodiment, the expression product is encoded by a sequence of nucleotides as set forth in SEQ ID NO:5 or a derivative homolog or analog thereof including a nucleotide sequence having at least about 50% identity to SEQ ID NO:5.

In still another particularly preferred embodiment, the expression product is encoded by a sequence of nucleotides as set forth in SEQ ID NO:6 or a derivative homolog or analog thereof including a nucleotide sequence having at least about 50% identity to SEQ ID NO:6.

Higher similarities are also contemplated by the present invention such as greater than 50% or 60% or 70% or 80% or 90% or 95% or 96% or 97% or 98% or 99% or above. Further examples include 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100%.

Another aspect of the present invention is directed to an isolated expression product that is differentially expressed in cells from a subject having one or more diseases and/or conditions wherein the protein is selected from the list consisting of:
(i) an expression product encoded by a nucleotide sequence substantially as set forth in SEQ ID NO:1 (CXS-740) or a derivative, homolog or analog thereof or a sequence encoding an amino acid sequence having at least about 50% similarity to this sequence or a derivative, homolog, analog, chemical equivalent or mimetic of said protein;
(ii) an expression product encoded by a nucleotide sequence substantially as set forth in SEQ ID NO:2 (CXS-741) or a derivative, homolog or analog thereof or a sequence encoding an amino acid sequence having at least about 50% similarity to this sequence or a derivative, homolog, analog, chemical equivalent or mimetic of said protein;
(ii) an expression product encoded by a nucleotide sequence substantially as set forth in SEQ ID NO:3 (CXS-742) or a derivative, homolog or analog thereof or a sequence encoding an amino acid sequence having at least about 50% similarity to this sequence or a derivative, homolog, analog, chemical equivalent or mimetic of said protein;
(iv) an expression product encoded by a nucleotide sequence substantially as set forth in SEQ ID NO:4 (CXS-743) or a derivative, homolog or analog thereof or a sequence encoding an amino acid sequence having at least about 50% similarity to this sequence or a derivative, homolog, analog, chemical equivalent or mimetic of said protein;
(v) an expression product encoded by a nucleotide sequence substantially as set forth in SEQ ID NO:5 (CXS-744) or a derivative, homolog or analog thereof or a sequence encoding an amino acid sequence having at least about 50% similarity to this sequence or a derivative, homolog, analog, chemical equivalent or mimetic of said protein;
(vi) an expression product encoded by a nucleotide sequence substantially as set forth in SEQ ID NO:6 (CXS-745) or a derivative, homolog or analog thereof or a sequence encoding an amino acid sequence having at least about 50% similarity to this sequence or a derivative, homolog, analog, chemical equivalent or mimetic of said protein;
(vii) an expression product encoded by a nucleic acid molecule capable of hybridizing to the nucleotide sequence as set forth in SEQ ID NO:1 (CXS-740) or a derivative, homolog or analog thereof;
(viii) an expression product encoded by a nucleic acid molecule capable of hybridizing to the nucleotide sequence as set forth in SEQ ID NO:2 (CXS-741) or a derivative, homolog or analog thereof;
(ix) an expression product encoded by a nucleic acid molecule capable of hybridizing to the nucleotide sequence as set forth in SEQ ID NO:3 (CXS-742) or a derivative, homolog or analog thereof;
(x) an expression product encoded by a nucleic acid molecule capable of hybridizing to the nucleotide sequence as set forth in SEQ ID NO:4 (CXS-743) or a derivative, homolog or analog thereof;
(xi) an expression product encoded by a nucleic acid molecule capable of hybridizing to the nucleotide sequence as set forth in SEQ ID NO:5 (CXS-744) or a derivative, homolog or analog thereof;
(xii) an expression product encoded by a nucleic acid molecule capable of hybridizing to the nucleotide sequence as set forth in SEQ ID NO:6 (CXS-745) or a derivative, homolog or analog thereof.

The protein of the present invention is preferably in isolated form. By “isolated” is meant a protein having undergone at least one purification step and this is conveniently defined, for example, by a composition comprising at least about 10% subject protein, preferably at least about 20%, more preferably at least about 30%, still more preferably at least about 40-50%, even still more preferably at least about 60-70%, yet even still more preferably 80-90% or greater, such as 10, 11, 12, 13, 14, 15, 16, 17, 18, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100% of subject protein relative to other components as determined by molecular weight, amino acid sequence or other convenient means. The protein of the present invention may also be considered, in a preferred embodiment, to be biologically pure.

The terms “sequence similarity” and “sequence identity” as used herein refers to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

The nucleotide sequence or amino acid sequence of the present invention may correspond to exactly the same sequence of the naturally occurring gene (or corresponding cDNA) or protein or may carry one or more nucleotide or amino acid substitutions, additions and/or deletions. The nucleotide sequences set forth in SEQ ID NO:1, SEQ ID NO:2 or SEQ ID NO:3 or SEQ ID NO:4 or SEQ ID NO:5 or SEQ ID NO:6 correspond to the genes referred to herein as CXS-740, CXS-741, CXS-742, CXS-743, CXS-744 and CXS-745, respectively. The corresponding expression products are CXS-740, CXS-741, CXS-742, CXS-743, CXS-744 and CXS-745, respectively. Reference herein to CXS-740, CXS-741, CXS-742, CXS-743, CXS-744 and CXS-745 includes, where appropriate, reference to the genomic gene or cDNA as well as any naturally occurring or induced derivatives. Apart from the substitutions, deletions and/or additions to the nucleotide sequence, the present invention further encompasses mutants, fragments, parts and portions of the nucleotide sequence corresponding to CXS-740, CXS-741, CXS-742, CXS-743, CXS-744 and CXS-745.

The identification of CXS-740, CXS-741, CXS-742, CXS-743, CXS-744 and CXS-745 permits the generation of a range of therapeutic molecules capable of modulating expression of CXS-740, CXS-741, CXS-742, CXS-743, CXS-744 and CXS-745 or modulating the activity of CXS-740, CXS-741, CXS-742, CXS-743, CXS-744 and CXS-745. Modulators contemplated by the present invention includes agonists and antagonists of CXS-740, CXS-741, CXS-742, CXS-743, CXS-744 and CXS-745 expression. Antagonists of CXS-740, CXS-741, CXS-742, CXS-743, CXS-744 and CXS-745 expression include antisense molecules, ribozymes and co-suppression molecules. Agonists include molecules which increase promoter activity or which interfere with negative regulatory mechanisms. Antagonists of CXS-740, CXS-741, CXS-742, CXS-743, CXS-744 and CXS-745 include antibodies and inhibitor peptide fragments. All such molecules may first need to be modified to enable such molecules to penetrate cell membranes. Alternatively, viral agents may be employed to introduce genetic elements to modulate expression of CXS-740, CXS-741, CXS-742, CXS-743, CXS-744 and CXS-745.

The present invention contemplates, therefore, a method for modulating expression of one or more of CXS-740, CXS-741, CXS-742, CXS-743, CXS-744 and CXS-745 in a mammal, said method comprising contacting the CXS-740, CXS-741, CXS-742, CXS-743, CXS-744 and CXS-745 gene with an effective amount of a modulator of CXS-740, CXS-741, CXS-742, CXS-743, CXS-744 and CXS-745 expression for a time and under conditions sufficient to up-regulate or down-regulate or otherwise modulate expression of CXS-740, CXS-741, CXS-742, CXS-743, CXS-744 and CXS-745. For example, a nucleic acid molecule encoding CXS-740, CXS-741, CXS-742, CXS-743, CXS-744 and CXS-745 or a derivative or homolog thereof may be introduced into a cell to enhance the ability of that cell to produce CXS-740, CXS-741, CXS-742, CXS-743, CXS-744 and CXS-745, conversely, CXS-740, CXS-741, CXS-742, CXS-743, CXS-744 and CXS-745 antisense sequences such as oligonucleotides may be introduced to decrease the availability of CXS-740, CXS-741, CXS-742, CXS-743, CXS-744 and CXS-745 molecules.

Another aspect of the present invention contemplates a method of modulating activity of CXS-740, CXS-741, CXS-742, CXS-743, CXS-744 and CXS-745 in a mammal, said method comprising administering to said mammal a modulating effective amount of a molecule for a time and under conditions sufficient to increase or decrease CXS-740, CXS-741, CXS-742, CXS-743, CXS-744 and CXS-745 activity. The molecule may be a proteinaceous molecule or a chemical entity and may also be a derivative of CXS-740, CXS-741, CXS-742, CXS-743, CXS-744 and CXS-745 or its ligand.

Modulating levels of CXS-740, CXS-741, CXS-742, CXS-743, CXS-744 and CXS-745 expression is important in the treatment of a range of conditions such as diabetes, complications associated with diabetes, such as blindness, nephropathy and/or cardiovascular disease, inflammation, mitochondrial dysfunction, myopathy, genetic disorder or cancer or in modulating apoptosis, signal transduction and/or nuclear targeting. The present invention has application in the treatment of humans as well as in the veterinary and animal husbandry industries. Accordingly, subjects contemplated for treatment in accordance with the present invention includes, but is not limited to humans, primates, livestock animals (e.g. pigs, sheep, cows, horses, donkeys), laboratory test animals (e.g. mice, rats, guinea pigs, hamsters, rabbits), companion animals (e.g. dogs, cats) and captured wild animals (e.g. foxes, kangaroos, deer). A particularly preferred host is a human, primate or livestock animal.

Accordingly, the present invention contemplates therapeutic and prophylactic uses of CXS-740, CXS-741, CXS-742, CXS-743, CXS-744 and/or CXS-745 amino acid and nucleic acid molecules in addition to CXS-740, CXS-741, CXS-742, CXS-743, CXS-744 and/or CXS-745 agonistic and antagonistic agents.

The present invention contemplates, therefore, a method of modulating expression of CXS-740, CXS-741, CXS-742, CXS-743, CXS-744 and/or CXS-745 in a mammal, said method comprising contacting the CXS-740, CXS-741, CXS-742, CXS-743, CXS-744 and/or CXS-745 genes with an effective amount of an agent for a time and under conditions sufficient to up-regulate, down-regulate or otherwise module expression of CXS-740, CXS-741, CXS-742, CXS-743, CXS-744 and/or CXS-745. For example, antisense sequences such as oligonucleotides may be utilized.

Conversely, nucleic acid molecules encoding CXS-740, CXS-741, CXS-742, CXS-743, CXS-744 and/or CXS-745 or derivatives thereof may be introduced to up-regulate one or more specific functional activities.

Another aspect of the present invention contemplates a method of modulating activity of CXS-740, CXS-741, CXS-742, CXS-743, CXS-744 and CXS-745 in a subject, said method comprising administering to said subject a modulating effective amount of an agent for a time and under conditions sufficient to increase or decrease CXS-740, CXS-741, CXS-742, CXS-743, CXS-744 and CXS-745 activity.

Still another aspect of the present invention provides a method of treating a subject suffering from diabetes or a complication thereof, said method comprising administering to said subject an effective amount of an agent for a time and under conditions sufficient to down-regulate the level or activity CXS-741 (SEQ ID NO:2) and/or CXS-744 (SEQ ID NO:5).

Even yet another aspect of the present invention contemplates a method of treating a subject suffering from an inflammatory condition said method comprising administering to said subject an effective amount of an agent for a time and under conditions sufficient to down-regulate the level of activity of CXS-741 (SEQ ID NO:2) and/or CXS-744 (SEQ ID NO:5).

Modulation of activity by the administration of an agent to a mammal can be achieved by one of several techniques, including but in no way limited to introducing into said mammal a proteinaceous or non-proteinaceous molecule which:
(i) modulates expression of CXS-740, CXS-741, CXS-742, CXS-743, CXS-744 and CXS-745;
(ii) functions as an antagonist of CXS-740, CXS-741, CXS-742, CXS-743, CXS-744 and CXS-745;
(iii) functions as an agonist of CXS-740, CXS-741, CXS-742, CXS-743, CXS-744 and CXS-745.

Yet another aspect of the present invention contemplates the use an isolated nucleic acid molecule comprising a sequence of nucleotides said nucleic acid molecule which has is differentially expressed in cells from a subject having one or more diseases and/or conditions wherein the isolated molecule is encoded by a nucleic acid molecule selected from the list consisting of:
(i) a nucleic acid molecule comprises a nucleotide sequence as set forth in SEQ ID NO:1 (CXS-740) or a nucleotide sequence having at least about 50% identity thereto or a nucleotide sequence capable of hybridizing to SEQ ID NO:1 (CXS-740) or its complementary form;
(ii) a nucleic acid molecule comprises a nucleotide sequence as set forth in SEQ ID NO:2 (CXS-741) or a nucleotide sequence having at least about 50% identity thereto or a nucleotide sequence capable of hybridizing to SEQ ID NO:2 (CXS-741) or its complementary form;
(iii) a nucleic acid molecule comprises a nucleotide sequence as set forth in SEQ ID NO:3 (CXS-742) or a nucleotide sequence having at least about 50% identity thereto or a nucleotide sequence capable of hybridizing to SEQ ID NO:3 (CXS-742) or its complementary form;
(iv) a nucleic acid molecule comprises a nucleotide sequence as set forth in SEQ ID NO:4 (CXS-743) or a nucleotide sequence having at least about 50% identity thereto or a nucleotide sequence capable of hybridizing to SEQ ID NO:4 (CXS-743) or its complementary form;
(v) a nucleic acid molecule comprises a nucleotide sequence as set forth in SEQ ID NO:5 (CXS-744) or a nucleotide sequence having at least about 50% identity thereto or a nucleotide sequence capable of hybridizing to SEQ ID NO:5 (CXS-744) or its complementary form;
(vi) a nucleic acid molecule comprises a nucleotide sequence as set forth in SEQ ID NO:6 (CXS-745) or a nucleotide sequence having at least about 50% identity thereto or a nucleotide sequence capable of hybridizing to SEQ ID NO:6 (CXS-745) or its complementary form;
   in the manufacture of a medicament for the treatment of one or more diseases and/or conditions.

The proteinaceous molecule may be derived from natural or recombinant sources including fusion proteins or following, for example, natural product screening or the screening of chemical libraries. The non-proteinaceous molecule may be, for example, a nucleic acid molecule or may be derived from natural sources, such as for example natural product screening or may be chemically synthesized. The present invention contemplates chemical analogs of CXS-740, CXS-741, CXS-742, CXS-743, CXS-744 and/or CXS-745 or small molecules capable of acting as agonists or antagonists. Chemical agonists may not necessarily be derived from CXS-740, CXS-741, CXS-742, CXS-743, CXS-744 and/or CXS-745 but may share certain conformational similarities. Alternatively, chemical agonists may be specifically designed to mimic certain physiochemical properties. Antagonists may be any compound capable of blocking, inhibiting or otherwise preventing CXS-740, CXS-741, CXS-742, CXS-743, CXS-744 and/or CXS-745 from carrying out their normal biological functions. Antagonists include monoclonal antibodies, antisense and sense nucleic acids which prevent transcription or translation of CXS-740, CXS-741, CXS-742, CXS-743, CXS-744 and CXS-745 genes or mRNA in mammalian cells. Modulation of expression may also be achieved utilizing antigens, RNA, RNAi, ribosomes, DNAzymes, RNA aptamers or antibodies.

The proteinaceous or non-proteinaceous molecule may act either directly or indirectly to modulate the expression of CXS-740, CXS-741, CXS-742, CXS-743, CXS-744 and/or CXS-745 or the activity of CXS-740, CXS-741, CXS-742, CXS-743, CXS-744 and/or CXS-745. Said molecule acts directly if it associates with CXS-740, CXS-741, CXS-742, CXS-743, CXS-744 and/or CXS-745 or CXS-740, CXS-741, CXS-742, CXS-743, CXS-744 and/or CXS-745 to modulate expression or activity. Said molecule acts indirectly if it associates with a molecule other than CXS-740, CXS-741, CXS-742, CXS-743, CXS-744 and/or CXS-745 or CXS-740, CXS-741, CXS-742, CXS-743, CXS-744 and/or CXS-745 which other molecule either directly or indirectly modulates the expression or activity of CXS-740, CXS-741, CXS-742, CXS-743, CXS-744 and/or CXS-745 or CXS-740, CXS-741, CXS-742, CXS-743, CXS-744 and/or CXS-745. Accordingly, the method of the present invention encompasses the regulation of CXS-740, CXS-741, CXS-742, CXS-743, CXS-744 and/or CXS-745 or CXS-740, CXS-741, CXS-742, CXS-743, CXS-744 and/or CXS-745 expression or activity via the induction of a cascade of regulatory steps.

The molecules which may be administered to a mammal in accordance with the present invention may also be linked to a targeting means such as a monoclonal or polyclonal antibody, which provides specific delivery of these molecules to the target cells.

A further aspect of the present invention relates to the use of the invention in relation to mammalian disease conditions. For example, the present invention is particularly useful but in no way limited to use in a therapeutic or prophylactic treatment of diabetes, complications associated with diabetes, such as blindness, nephropathy and/or cardiovascular disease, inflammation, mitochondrial dysfunction, myopathy, genetic disorder or cancer or in modulating apoptosis, signal transduction and/or nuclear targeting.

Accordingly, another aspect of the present invention relates to a method of treating a mammal suffering from a condition characterized by one or more symptoms of diabetes, complications associated with diabetes, such as blindness, nephropathy and/or cardiovascular disease, inflammation, mitochondrial dysfunction, myopathy, genetic disorder or cancer or in modulating apoptosis, signal transduction and/or nuclear targeting, said method comprising administering to said mammal an effective amount of an agent for a time and under conditions sufficient to modulate the expression of CXS-740, CXS-741, CXS-742, CXS-743, CXS-744 and/or CXS-745 or sufficient to modulate the activity of CXS-740, CXS-741, CXS-742, CXS-743, CXS-744 and/or CXS-745.

In another aspect, the present invention relates to a method of treating a mammal suffering from a disease condition characterized by one or more symptoms of diabetes, complications associated with diabetes, such as blindness, nephropathy and/or cardiovascular disease, inflammation, mitochondrial dysfunction, myopathy, genetic disorder or cancer or in modulating apoptosis, signal transduction and/or nuclear targeting, said method comprising administering to said mammal an effective amount of CXS-740, CXS-741, CXS-742, CXS-743, CXS-744 and CXS-745 or CXS-740, CXS-741, CXS-742, CXS-743, CXS-744 and/or CXS-745.

As used herein “myopathy” refers to any abnormal conditions or disease of the muscle tissues, which include the muscles over our bones (skeletal muscle) and the heart (cardiac muscle).

Mitochondrial dysfunction relates to abnormalities in mitochondria. Mitochondria are part of the cell (organelle) that is responsible for energy production. The organelle consists of two sets of membranes, a smooth continuous outer coat and an inner membrane arranged in tubules or in folds that form plate-like double membranes (cristae). Mitochondria are the principal energy source of the cell, containing the cytochrome enzymes of terminal electron transport and the enzymes of the citric acid cycle, fatty acid oxidation, and oxidative phosphorylation. They are responsible for converting nutrients into energy as well as many other specialized tasks. Mitochondria are complex organelles located in virtually all cells of the body. A large degree of their complexity is due to the fact that over 1000 proteins are located in the mitochondria. Thirteen of these proteins are encoded by the mitochondrial DNA (mtDNA), while the remainder are nuclear-encoded, and imported into the mitochondria.

Symptoms of mitochondrial dysfunction include weakness (which may be intermittent), neuropathic pain, absent reflexes, gastrointestinal problem (gastroesophogeal reflux, delayed gastric emptying, constipation, pseudo-obstruction), fainting, absent or excessive sweating resulting in temperature regulation problems, hypotonia, cramping and muscle pain, proximal renal tubular wasting resulting in loss of protein, magnesium, phosphorous, calcium and other electrolytes, cardiac conduction defects (heart blocks) and cardiomyopathy, hypoglycemia (low blood sugar) and liver failure, visual loss and blindness, hearing loss and deafness, and diabetes and exocrine pancreatic failure (inability to make digestive enzymes).

There may also be systemic problems associated with mitochondrial dysfunction, including failure to gain weight, short stature, fatigue, respiratory problems.

Mitochondrial defects have been linked to Alzheimer's, Parkinson's, diabetes, autism, and the aging process. Other disease associated with mitochondrial dysfunction include, LIC (Lethal Infantile Cardiomyopathy), Beta-oxidation Defects, COX Deficiency, Mitochondrial Cytopathy, Alpers Disease, Barth syndrome, Carnitine-Acyl-Carnitine Deficiency, Carnitine Deficiency, Co-Enzyme Q10 Deficiency, Complex I Deficiency, Complex II Deficiency, Complex III Deficiency, Complex IV Deficiency, Complex V Deficiency, CPEO, CPT I Deficiency, Glutaric Aciduria Type II, KSS, lactic acidosis, LCAD, LCHAD, Leigh Disease, LHON, Luft Disease, MAD, MCA, MELAS, MERRF, mitochondrial DNA depletion, Mitochondrial Encephalopathy, MNGIE, NARP, Pearson Syndrome, Pyruvate Carboxylase Deficiency, Pyruvate Dehydrogenase Deficiency, SCAD, SCHAD and VLCAD.

Alpers Disease, or Progressive Infantile Poliodystrophy, includes symptoms such as seizures, dementia, spasticity, blindness, liver dysfunction, and cerebral degeneration. (Luft; Proceedings of the National Academy of Sciences of the United States of America; 91 (19): 8731-8, 1994).

Barth syndrome or LIC (Lethal Infantile Cardiomyopathy) is an X-linked recessive disorder the symptoms of which include skeletal myopathy, cardiomyopathy, short stature, and neutropenia. (Christodoulou; American Journal of Medical Genetics; 50 (3):255-64, 1994).

Carnitine-Acyl-Carnitine Deficiency is an autosomal recessive disorder, the symptoms of which are seizures, apnea, bradycardia, vomiting, lethargy, coma, enlarged liver, limb weakness, myoglobin in the urine, Reye-like symptoms triggered by fasting.

Carnitine Deficiency is an autosomal recessive disease, the symptoms of which include Cardiomyopathy, failure to thrive, and altered consciousness or coma, sometimes hypotonia.

Co-Enzyme Q10 Deficiency is most likely an autosomal recessive disease, the symptoms of which include Encephalomyopathy, mental retardation, exercise intolerance, ragged-red fibers, and recurrent myoglobin in the urine.

Complex I Deficiency or NADH dehydrogenase (NADH-CoQ reductase) deficiency is an autosomal disease, the symptoms of which are classified by three major forms: (1) fatal infantile multisystem disorder, characterized by developmental delay, muscle weakness, heart disease, congenital lactic acidosis, and respiratory failure; (2) myopathy beginning in childhood or in adult life, manifesting as exercise intolerance or weakness. Elevated lactic acid common; and (3) mitochondrial encephalomyopathy (including MELAS), which may begin in childhood or adult life and consists of variable combinations of symptoms and signs, including ophthalmoplegia, seizures, dementia, ataxia, hearing loss, pigmentary retinopathy, sensory neuropathy, and uncontrollable movements. In addition, this disorder may cause Leigh Syndrome.

Complex II Deficiency or Succinate dehydrogenase deficiency, the symptoms of which include encephalomyopathy and various manifestations, including failure to thrive, developmental delay, hyoptonia, lethargy, respiratory failure, ataxia, myoclonus and lactic acidosis. May also cause Leigh Syndrome.

Complex III Deficiency or Ubiquinone-cytochrome c oxidoreductase deficiency, symptoms of which are categorized in four major forms: (1) fatal infantile encephalomyopathy, congenital lactic acidosis, hypotonia, dystrophic posturing, seizures, and coma. Ragged-red fibers common; (2) encephalomyopathies of later onset (childhood to adult life): various combinations of weakness, short stature, ataxia, dementia, hearing loss, sensory neuropathy, pigmentary retinopathy, and pyramidal signs. Ragged-red fibers common. Possible lactic acidosis; (3) myopathy, with exercise intolerance evolving into fixed weakness. Ragged-red fibers common. Possible lactic acidosis; and (4) infantile histiocytoid cardiomyopathy.

4Complex IV Deficiency or Cytochrome c oxidase deficiency is caused by a defect in Complex IV of the respiratory chain, the symptoms of which can be categorized in two major forms: (1) encephalomyopathy, which is typically normal for the first 6 to 12 months of life and then show developmental regression, ataxia, lactic acidosis, optic atrophy, ophthalmoplegia, nystagmus, dystonia, pyramidal signs, respiratory problems and frequent seizures; and (2) myopathy: Two main variants: (a) Fatal infantile myopathy: may begin soon after birth and accompanied by hypotonia, weakness, lactic acidosis, ragged-red fibers, respiratory failure, and kidney problems: and (b) Benign infantile myopathy: may begin soon after birth and accompanied by hypotonia, weakness, lactic acidosis, ragged-red fibers, respiratory problems, but (if the child survives) followed by spontaneous improvement.

Complex V Deficiency or ATP synthase deficiency includes symptoms such as slow, progressive myopathy.

CPEO or Chronic Progressive External Ophthalmoplegia Syndrome includes symptoms such as visual myopathy, retinitis pigmentosa, dysfunction of the central nervous system. It is caused by single mitochondrial DNA deletions, with Mitochondrial DNA point mutation, A3243G being the most common (Luft; 1994 Supra).

CPT I Deficiency is an autosomal recessive disease and includes symptoms such as enlarged liver and recurrent Reye-like episodes triggered by fasting or illnesses.

CPT II Deficiency is an autosomal recessive disease, the symptoms of which include exercise intolerance, fasting intolerance, muscle pain, muscle stiffness, and myoglobin in the urine and in infants, Reye-like syndrome, enlarged liver, hypoglycemia, enlarged heart and cardiac arrhythmia.

KSS or Kearns-Sayre Syndrome, in most cases is caused by large mitochondria DNA deletions. Symptoms associated with KSS include progressive external ophthalmoplegia, pigmentary retinopathy, heart block, and high cerebrospinal protein.

Lactic Acidosis is associated with the accumulation of lactic acid due to its production exceeding its use. Chronic lactic acidosis is a common symptom of mitochondrial disease.

LCAD or Long-Chain Acyl-CoA Dehydrogenase Deficiency, is an autosomal recessive disorder, which causes a fatal syndrome, in infants, typified by failure to thrive, enlarged liver, enlarged heart, metabolic encephalopathy and hypotonia.

LCHAD is an autosomal recessive disorder, characterized by symptoms such as encephalopathy, liver dysfunction, cardiomyopathy, and myopathy. Also pigmentary retinopathy and peripheral neuropathy.

Leigh Disease or Syndrome or Subacute Necrotizing Encephalomyelopathy is characterized by symptoms such as Seizures, hypotonia, fatigue, nystagmus, poor reflexes, eating and swallowing difficulties, breathing problems and poor motor function.

LHON or Leber Hereditary Optic Neuropathy is caused by mitochondrial DNA point mutations, including G14459A, among others. Symptoms associated with LHON include primarily blindness in young men. Less common symptoms include mild dementia, ataxia, spasticity, peripheral neuropathy and heart conduction defects.

Luft Disease is characterized by symptoms such as hypermetabolism, with fever, heat intolerance, profuse perspiration, polyphagia, polydipsia, ragged-red fibers, and resting tachycardia. In addition to exercise intolerance with mild weakness.

MAD or Glutaric Aciduria Type II or multiple Acyl-CoA Dehydrogenase Deficiency is caused by defects of the flavoproteins responsible for transferring electrons (ETF or ETF-dehydrogenase) therefore affecting the function of all six ETF-funneling acyl-CoA dehydrogenases

MCAD or Medium-Chain Acyl-CoA Dehydrogenase Deficiency is an autosomal recessive disorder, which afflicts infants or young children with episodes of encephalopathy, enlarged and fatty degeneration of the liver, and low carnitine in the blood.

MELAS or Mitochondrial Encephalomyopathy Lactic Acidosis and Strokelike Episodes is caused by mitochondrial DNA point mutations, the most common of which is A3243G. It is characterized by symptoms: Short stature, seizures, stroke-like episodes with focused neurological deficits, recurrent headaches, cognitive regression, disease progression ragged-red fibers (Koo, et. al.; Annals of Neurology; 34 (1):25-32, 1993).

MERRF or Myoclonic Epilepsy and Ragged-Red Fiber Disease is caused by the mitochondrial DNA point mutations A8344G and T8356C. Its symptoms include myoclonus, epilepsy, progressive ataxia, muscle weakness and degeneration, deafness and dementia (Luft; 1994 Supra).

There are three forms of mitochondrial DNA Depletion. These include: (1) congenital myopathy: Neonatal weakness, hypotonia requiring assisted ventilation, possible renal dysfunction. Severe lactic acidosis. Prominent ragged-red fibers. Death due to respiratory failure usually occurs prior to one year of age; (2) infantile myopathy: Following normal early development until one year old, weakness appears and worsens rapidly, causing respiratory failure and death typically within a few years; and (3) hepatopathy, enlarged liver and intractable liver failure, myopathy. Severe lactic acidosis. Death is typical within the first year.

Mitochondrial Encephalopathy also includes Encephalomyopathy and Encephalomyelopathy.

MNGIE or Myoneurogastrointestinal Disorder and Encephalopathy, include symptoms such as progressive external ophthalmoplegia, limb weakness, peripheral neuropathy, digestive tract disorders, leukodystrophy, lactic acidosis and ragged red fibers.

NARP or Neuropathy, Ataxia, and Retinitis Pigmentosa is caused by mitochondrial DNA point mutations in genes associated with Complex V, including T8993G, (also T8993C by some researchers). Leigh Syndrome may result if the percentage of mutation is high enough.

Pearson Syndrome is characterized by symptoms associated with bone marrow and pancreas dysfunction. It is caused by single mitochondrial DNA deletions. Inheritance is usually sporadic. Those who survive infancy usually develop Kearns-Sayre Syndrome.

Pyruvate Carboxylase Deficiency is an autosomal recessive disorder, the symptoms of which include lactic acidosis, hypoglycemia, severe retardation, failure to thrive, in addition to seizures and spasticity.

Pyruvate Dehydrogenase Deficiency is characterized by symptoms such as lactic acidosis, ataxia, pyruvic acidosis, spinal and cerebellar degeneration. Less common symptoms include agenesis of the corpus callosum and lesions in the basal ganglia, cerebellum, and brain stem, growth delay, hypotonia, seizures and polyneuropathy.

SCAD or Short-Chain Acyl-CoA Dehydrogenase Deficiency, is an autosomal recessive disorder characterized by symptoms such as failure to thrive, developmental delay and hypoglycemia.

SCHAD is an autosomal recessive disorder, characterized by encephalopathy and possibly liver disease or cardiomyopathy.

VLCAD or Very Long-Chain Acyl-CoA Dehydrogenase Deficiency is an autosomal recessive disorder, characterized by various manifestations, ranging from fatal infantile encephalopathy to recurrent myoglobin in the urine, similar to the myopathic form of CPT II deficiency.

An “effective amount” means an amount necessary at least partly to attain the desired physiological effect or to delay the onset or inhibit progression or halt altogether, the onset or progression of a particular condition of the individual to be treated, the taxonomic group of the individual to be treated, the degree of protection desired, the formulation of the vaccine, the assessment of the medical situation, and other relevant factors. It is expected that the amount will fall in a relatively broad range that can be determined through routine trials.

In accordance with these methods, CXS-740, CXS-741, CXS-742, CXS-743, CXS-744 and/or CXS-745 or CXS-740, CXS-741, CXS-742, CXS-743, CXS-744 and CXS-745 or agents capable of modulating the expression or activity of said molecules may be co-administered with one or more other compounds or other molecules. By “co-administered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

In yet another aspect, the present invention relates to the use of an agent capable of modulating the expression of CXS-740, CXS-741, CXS-742, CXS-743, CXS-744 and/or CXS-745 or a derivative, homolog or analog thereof in the manufacture of a medicament for the treatment of a condition characterized by diabetes, complications associated with diabetes, such as blindness, nephropathy and/or cardiovascular disease, inflammation, mitochondrial dysfunction, myopathy, genetic disorder or cancer.

In still yet another aspect, the present invention relates to the use of an agent capable of modulating the activity of CXS-740, CXS-741, CXS-742, CXS-743, CXS-744 and/or CXS-745 or a derivative, homolog, analog, chemical equivalent or mimetic thereof in the manufacture of a medicament for the treatment of a condition characterized by diabetes, complications associated with diabetes, such as blindness, nephropathy and/or cardiovascular disease, inflammation, mitochondrial dysfunction, myopathy, genetic disorder or cancer or in modulating apoptosis, signal transduction and/or nuclear targeting.

A further aspect of the present invention relates to the use of CXS-740, CXS-741, CXS-742, CXS-743, CXS-744 and/or CXS-745 or derivative, homolog or analog thereof or CXS-740, CXS-741, CXS-742, CXS-743, CXS-744 and/or CXS-745 or derivative, homolog, analog, chemical equivalent or mimetic thereof in the manufacture of a medicament for the treatment of a condition characterized by diabetes, complications associated with diabetes, such as blindness, nephropathy and/or cardiovascular disease, inflammation, mitochondrial dysfunction, myopathy, genetic disorder or cancer or in modulating apoptosis, signal transduction and/or nuclear targeting.

Still yet another aspect of the present invention relates to agents for use in modulating the expression of CXS-740, CXS-741, CXS-742, CXS-743, CXS-744 and/or CXS-745 or a derivative, homolog or analog thereof.

A further aspect relates to agents for use in modulating CXS-740, CXS-741, CXS-742, CXS-743, CXS-744 and/or CXS-745 activity or a derivative, homolog, analog, chemical equivalent or mimetic thereof.

Still another aspect of the present invention relates to CXS-740, CXS-741, CXS-742, CXS-743, CXS-744 and/or CXS-745 or derivative, homolog or analog thereof or CXS-740, CXS-741, CXS-742, CXS-743, CXS-744 and/or CXS-745 or derivative, homolog, analog, chemical equivalent or mimetic thereof for use in treating a condition characterized by one or more symptoms of diabetes, complications associated with diabetes, such as blindness, nephropathy and/or cardiovascular disease, inflammation, mitochondrial dysfunction, myopathy, genetic disorder or cancer or in modulating apoptosis, signal transduction and/or nuclear targeting.

In a related aspect of the present invention, the mammal undergoing treatment may be a human or an animal in need of therapeutic or prophylactic treatment.

The terms “treating” and “treatment” as used herein refer to a reduction in the severity and/or frequency of symptoms associated with inter alia diabetes, complications associated with diabetes, such as blindness, nephropathy and/or cardiovascular disease, inflammation, mitochondrial dysfunction, myopathy, genetic disorder or cancer or in modulating apoptosis, signal transduction and/or nuclear targeting, elimination of symptoms and/or the underlying cause, prevention of the occurrence of symptoms of disease and/or the underlying cause and improvement or remediation of damage.

“Treating” a subject may involve prevention of the disorder or disease condition or adverse physiological event in a susceptible individual as well as treatment of a clinically symptomatic individual by inhibiting a disease or disorder. Generally, such conditions involve, weakness (which may be intermittent), neuropathic pain, absent reflexes, gastrointestinal problem (gastroesophogeal reflux, delayed gastric emptying, constipation, pseudo-obstruction), fainting, absent or excessive sweating resulting in temperature regulation problems weakness, hypotonia, cramping, muscle pain, proximal renal tubular wasting resulting in loss of protein, magnesium, phosphorous, calcium and other electrolytes, cardiac conduction defects (heart blocks) and cardiomyopathy, hypoglycemia (low blood sugar) and liver failure, visual loss and blindness, hearing loss and deafness, diabetes and exocrine pancreatic failure (inability to make digestive enzymes), mitochondrial dysfunction, including failure to gain weight, short statue, fatigue and respiratory problems.

Accordingly, the present invention contemplates in one embodiment a composition comprising a modulator of CXS-740, CXS-741, CXS-742, CXS-743, CXS-744 and CXS-745 expression or CXS-740, CXS-741, CXS-742, CXS-743, CXS-744 and CXS-745 activity and one or more pharmaceutically acceptable carriers and/or diluents. In another embodiment, the composition CXS-740, CXS-741, CXS-742, CXS-743, CXS-744 and CXS-745 or a derivative, homolog, analog or mimetic thereof and one or more pharmaceutically acceptable carriers and/or diluents.

For brevity, all such components of such a composition are referred to as “active components”.

The compositions of active components in a form suitable for injectable use include sterile aqueous solutions (where water soluble) and sterile powders for the extemporaneous preparation of sterile injectable solutions. In all cases, the form must be sterile and must be fluid to the extent that easy syringability exists. It must be stable under the conditions of manufacture and storage and must be preserved against the contaminating action of microorganisms such as bacteria and fungi.

The carrier can be a solvent or other medium containing, for example, water, ethanol, polyol (for example, glycerol, propylene glycol and liquid polyethylene glycol, and the like), suitable mixtures thereof, and vegetable oils.

The preventions of the action of microorganisms can be brought about by various antibacterial and antifungal agents, for example, parabens, chlorobutanol, phenol, sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active components in the required amount in the appropriate solvent with optionally other ingredients, as required, followed by sterilization by, for example, filter sterilization, irradiation or other convenient means. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When CXS-740, CXS-741, CXS-742, CXS-743, CXS-744 and CXS-745 and CXS-740, CXS-741, CXS-742, CXS-743, CXS-744 and CXS-7452 CXS-740, CXS-741, CXS-742, CXS-743, CXS-744 and CXS-745 itself are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations.

Pharmaceutically acceptable carriers and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active component may be compounded for convenient and effective administration in sufficient amounts with a suitable pharmaceutically acceptable carrier in dosage unit form. A unit dosage form can, for example, contain the principal active component in amounts ranging from 0.5 μg to about 2000 mg. Expressed in proportions, the active compound is generally present in from about 0.5 μg to about 2000 mg/ml of carrier. In the case of compositions containing supplementary active ingredients, the dosages are determined by reference to the usual dose and manner of administration of the said ingredients.

In general terms, effective amounts of CXS-740, CXS-741, CXS-742, CXS-743, CXS-744 and CXS-745 will range from 0.01 ng/kg/body weight to above 10,000 mg/kg/body weight. Alternative amounts range from 0.1 ng/kg/body weight is above 1000 mg/kg/body weight. CXS-740, CXS-741, CXS-742, CXS-743, CXS-744 and CXS-745 may be administered per minute, hour, day, week, month or year depending on the condition being treated. The route of administration may vary and includes intravenous, intraperitoneal, sub-cutaneous, intramuscular, intranasal, via suppository, via infusion, via drip, orally or via other convenient means.

The pharmaceutical composition may also comprise genetic molecules such as a vector capable of transfecting target cells where the vector carries a nucleic acid molecule capable of modulating CXS-740, CXS-741, CXS-742, CXS-743, CXS-744 and CXS-745 expression or CXS-740, CXS-741, CXS-742, CXS-743, CXS-744 and CXS-745 activity. The vector may, for example, be a viral vector.

Still another aspect of the present invention is directed to antibodies CXS-740, CXS-741, CXS-742, CXS-743, CXS-744 and CXS-745 and their derivatives and homologs. Such antibodies may be monoclonal or polyclonal and may be selected from naturally occurring antibodies to CXS-740, CXS-741, CXS-742, CXS-743, CXS-744 and CXS-745 or may be specifically raised to CXS-740, CXS-741, CXS-742, CXS-743, CXS-744 and CXS-745 or derivatives or homologs thereof. In the case of the latter, CXS-740, CXS-741, CXS-742, CXS-743, CXS-744 and CXS-745 or their derivatives or homologs may first need to be associated with a carrier molecule. The antibodies and/or recombinant CXS-740, CXS-741, CXS-742, CXS-743, CXS-744 and CXS-745 or their derivatives of the present invention are particularly useful as therapeutic or diagnostic agents.

For example, CXS-740, CXS-741, CXS-742, CXS-743, CXS-744 and CXS-745 and their derivatives can be used to screen for naturally occurring antibodies to CXS-740, CXS-741, CXS-742, CXS-743, CXS-744 and CXS-745 which may occur in certain autoimmune diseases or where cell death is occurring. These may occur, for example in some autoimmune diseases. Alternatively, specific antibodies can be used to screen for CXS-740, CXS-741, CXS-742, CXS-743, CXS-744 and CXS-745. Techniques for such assays are well known in the art and include, for example, sandwich assays and ELISA.

Antibodies to CXS-740, CXS-741, CXS-742, CXS-743, CXS-744 and CXS-745 of the present invention may be monoclonal or polyclonal and may be selected from naturally occurring antibodies to the CXS-740, CXS-741, CXS-742, CXS-743, CXS-744 and CXS-745 or may be specifically raised to the CXS-740, CXS-741, CXS-742, CXS-743, CXS-744 and CXS-745 or their derivatives. In the case of the latter, the CXS-740, CXS-741, CXS-742, CXS-743, CXS-744 and CXS-745 protein may need first to be associated with a carrier molecule. Alternatively, fragments of antibodies may be used such as Fab fragments. Furthermore, the present invention extends to recombinant and synthetic antibodies and to antibody hybrids. A “synthetic antibody” is considered herein to include fragments and hybrids of antibodies. The antibodies of this aspect of the present invention are particularly useful for immunotherapy and may also be used as a diagnostic tool or as a means for purifying CXS-740, CXS-741, CXS-742, CXS-743, CXS-744 and CXS-745.

For example, specific antibodies can be used to screen for CXS-740, CXS-741, CXS-742, CXS-743, CXS-744 and CXS-745 proteins. The latter would be important, for example, as a means for screening for levels of CXS-740, CXS-741, CXS-742, CXS-743, CXS-744 and CXS-745 in a cell extract or other biological fluid or purifying CXS-740, CXS-741, CXS-742, CXS-743, CXS-744 and CXS-745 made by recombinant means from culture supernatant fluid. Techniques for the assays contemplated herein are known in the art and include, for example, sandwich assays and ELISA.

It is within the scope of this invention to include any second antibodies (monoclonal, polyclonal or fragments of antibodies) directed to the first mentioned antibodies discussed above. Both the first and second antibodies may be used in detection assays or a first antibody may be used with a commercially available anti-immunoglobulin antibody. An antibody as contemplated herein includes any antibody specific to any region of CXS-740, CXS-741, CXS-742, CXS-743, CXS-744 and CXS-745.

Both polyclonal and monoclonal antibodies are obtainable by immunization with the enzyme or protein and either Type Is utilizable for immunoassays. The methods of obtaining both types of sera are well known in the art. Polyclonal sera are less preferred but are relatively easily prepared by injection of a suitable laboratory animal with an effective amount of CXS-740, CXS-741, CXS-742, CXS-743, CXS-744 and CXS-745, or antigenic parts thereof, collecting serum from the animal, and isolating specific sera by any of the known immunoadsorbent techniques. Although antibodies produced by this method are utilizable in virtually any type of immunoassay, they are generally less favoured because of the potential heterogeneity of the product.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art. (See, for example, Douillard and Hoffman, Compendium of Immunology Vol. II, ed. by Schwartz, 1981; Kohler and Milstein, Nature 256: 495-499, 1975; Kohler and Milstein, European Journal of Immunology 6: 511-519, 1976).

Another aspect of the present invention contemplates a method for CXS-740, CXS-741, CXS-742, CXS-743, CXS-744 and CXS-745 or a derivative or homolog thereof in a biological sample from a subject, said method comprising contacting said biological sample with an antibody specific CXS-740, CXS-741, CXS-742, CXS-743, CXS-744 and CXS-745 or their antigenic derivatives or homologs for a time and under conditions sufficient for a complex to form, and then detecting said complex.

The presence of the complex is indicative of the presence CXS-740, CXS-741, CXS-742, CXS-743, CXS-744 and CXS-745. This assay may be quantitated or semi-quantitated to determine a propensity to develop mitochondrial dysfunction, myopathy, genetic disorder or cancer or in modulating apoptosis, signal transduction and/or nuclear targeting or to monitor a therapeutic regimen.

The presence of CXS-740, CXS-741, CXS-742, CXS-743, CXS-744 and CXS-745 may be accomplished in a number of ways such as by Western blotting and ELISA procedures. A wide range of immunoassay techniques are available as can be seen by reference to U.S. Pat. Nos. 4,016,043, 4,424,279 and 4,018,653. These, of course, includes both single-site and two-site or “sandwich” assays of the non-competitive types, as well as in the traditional competitive binding assays. These assays also include direct binding of a labeled antibody to a target.

Sandwich assays are among the most useful and commonly used assays. A number of variations of the sandwich assay technique exist, and all are intended to be encompassed by the present invention. Briefly, in a typical forward assay, an unlabeled antibody is immobilized on a solid substrate and the sample to be tested brought into contact with the bound molecule. After a suitable period of incubation, for a period of time sufficient to allow formation of an antibody-CXS-740, CXS-741, CXS-742, CXS-743, CXS-744 and CXS-745 complex, a second antibody specific to the CXS-740, CXS-741, CXS-742, CXS-743, CXS-744 and CXS-745, labeled with a reporter molecule capable of producing a detectable signal, is then added and incubated, allowing time sufficient for the formation of another complex of antibody-CXS-740, CXS-741, CXS-742, CXS-743, CXS-744 and CXS-745-labeled antibody. Any unreacted material is washed away, and the presence of CXS-740, CXS-741, CXS-742, CXS-743, CXS-744 and CXS-745 is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labeled antibody are added simultaneously to the bound antibody. These techniques are well known to those skilled in the art, including any minor variations as will be readily apparent. In accordance with the present invention, the sample is one which might contain CXS-740, CXS-741, CXS-742, CXS-743, CXS-744 and CXS-745 including cell extract, tissue biopsy or possibly serum, saliva, mucosal secretions, lymph, tissue fluid and respiratory fluid. The sample is, therefore, generally a biological sample comprising biological fluid but also extends to fermentation fluid and supernatant fluid such as from a cell culture.

The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g. from room temperature to about 37° C.) to allow binding of any subunit present in the antibody. Following the incubation period, the antibody subunit solid phase is washed and dried and incubated with a second antibody specific for a portion of CXS-740, CXS-741, CXS-742, CXS-743, CXS-744 and CXS-745. The second antibody is linked to a reporter molecule which is used to indicate the binding of the second antibody to CXS-740, CXS-741, CXS-742, CXS-743, CXS-744 and CXS-745.

An alternative method involves immobilizing the target molecules in the biological sample and then exposing the immobilized target to specific antibody which may or may not be labeled with a reporter molecule. Depending on the amount of target and the strength of the reporter molecule signal, a bound target may be detectable by direct labeling with the antibody. Alternatively, a second labeled antibody, specific to the first antibody is exposed to the target-first antibody complex to form a target-first antibody-second antibody tertiary complex. The complex is detected by the signal emitted by the reporter molecule.

By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, β-galactosidase and alkaline phosphatase, amongst others. The substrates to be used with the specific enzymes are generally chosen for the production, upon hydrolysis by the corresponding enzyme, of a detectable colour change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample. A “reporter molecule” also extends to use of cell agglutination or inhibition of agglutination such as red blood cells on latex beads, and the like.

Alternately, fluorescent compounds, such as fluorecein and rhodamine, may be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic colour visually detectable with a light microscope. As in the EIA, the fluorescent labeled antibody is allowed to bind to the first antibody-hapten complex. After washing off the unbound reagent, the remaining tertiary complex is then exposed to the light of the appropriate wavelength the fluorescence observed indicates the presence of the hapten of interest. Immunofluorescene and EIA techniques are both very well established in the art and are particularly preferred for the present method. However, other reporter molecules, such as radioisotope, chemiluminescent or bioluminescent molecules, may also be employed.

The present invention also contemplates genetic assays such as involving PCR analysis to detect CXS-740, CXS-741, CXS-742, CXS-743, CXS-744 and CXS-745 or their derivatives.

The assays of the present invention may also extend to measuring CXS-740, CXS-741, CXS-742, CXS-743, CXS-744 and CXS-745 or CXS-740, CXS-741, CXS-742, CXS-743, CXS-744 and CXS-745 in association with another gene or molecule.

The present invention is further described by the following non-limiting Examples.

### EXAMPLE 1

### Psammomys obesus Colony

A Psammomys obesus colony is maintained at Deakin University, Waurn Ponds, Geelong, Victoria, Australia with the breeding pairs fed ad libitum a diet of lucerne and standard laboratory chow. Animals are weaned at four weeks of age and sustained on a diet of standard laboratory chow from which 12% of energy was derived from fat, 63% from carbohydrate and 25% from protein (Barastoc, Pakenham, Australia). Animals are housed in a humidity and temperature controlled room (22±1° C.) with a 12-12-hour light-dark cycle.

Group A animals are lean, normoglycemic and normoinsulinemic (NGT), group B animals are obese, normoglycemic and hyperinsulinemic (IGT), and group C animals are obese, hyperglycemic and hyperinsulinemia (type 2 diabetic).

### EXAMPLE 2

### Analytical Methods

Whole blood glucose was measured immediately using an enzymatic glucose analyser (Model 27, Yellow Springs Instruments, OH). Plasma insulin concentrations were determined using a double antibody solid phase radioimmunoassay (Phadeseph, Kabi Pharmacia Diagnostics, Sweden).

### EXAMPLE 3

### RNA Extraction

Total RNA was extracted from tissue in a two-step process utilising Trizol (Invitrogen Life Technologies, Carlsbad, USA) and RNeasy (Qiagen, Hilden, Germany) protocols. The tissue samples were homogenised in Trizol (Invitrogen Life Technologies) and 1/5^{th} volume chloroform was added to the homogenate, which was then mixed and incubated at room temperature for 3 min. The homogenates were then separated by centrifugation at 13000×g for 15 min (4° C.). Following centrifugation the aqueous supernatant was removed and added to an equal volume of 70% ethanol. The solution was mixed and the RNA extracted using RNeasy kit spin columns (Qiagen) according to manufacturer's instructions. The RNA was eluted using RNAase free water and total RNA integrity, quantity and concentration was assessed using the RNA 6000 Nano Assay (Agilent Technologies, Palo Alto, USA) with the Agilent 2100 Bioanalyser (Agilent Technologies) as per the manufacturer's instructions. This system utilises capillary electrophoresis to separate and detect nucleic acid fragments by size through the interconnected micro channels on a Nano chip (Agilent Technologies). Good quality RNA was signified by an electropherogram displaying a marker peak, and two ribosomal peaks of which the 18 s band is at an approximate ratio of 1:2 to the 28 s band.

### EXAMPLE 4

### Statistical Analysis

All data are expressed as mean±S.E.M. A one-way analysis of variance in combination with post hoc least significant difference or Games-Howell test were used to compare means between groups, and t-tests were used where appropriate. A 2-tailed Pearson correlation was performed to analyse relationships between gene expression and phenotypes. Blood glucose and plasma insulin concentrations were log transformed prior to analysis to approximate a normal distribution. Differences were considered significant at P<0.05.

### EXAMPLE 5

### Signal Sequence Trap

Total RNA was extracted as described in Example 3 from red gastrocnemius muscle of lean, normoglycaemic, normoinsulinaemic (NGT), obese normoglycaemic, hyperinsulinaemic (IGT) and obese type 2 diabetic (T2D) P. obesus in the fed and fasted states. Equal amounts of RNA from each group were pooled and the mRNA extracted using an oligo(dT) cellulose column.

The mRNA was reverse transcribed using random 9mer primers to enrich for the 5′ end of mRNA. The random 9mer primers were engineered to contain a Not I restriction site (underlined) which was used for cloning:

| | |
|---|---|
| pTCTAGATCGCGAGCGGCCGCCCNNNNNNNNN | (SEQ ID NO: 7) |

After second strand synthesis, Sal I adapter addition and Not I digestion, the cDNA was run on a 1.5% w/v TBE agarose gel. The 300 to 800 base pair products were cut out of the gel, purified and quantitated using standard methodologies.

The Not I-Sal I digested skeletal muscle library was ligated into the retrovirus plasmid vector pLNCX2, 5′ to a murine interleukin 3 (mIL-3) gene that was engineered to lack a signal sequence. Transformation conditions were according to standard methodologies. Transformation of bacterial cells generated approximately 200,000 transformants. The plasmid library obtained from the 200,000 transformants was transfected into a retrovirus packaging cell line (293 Plat E).

The retrovirus library that was produced was used to infect the mIL-3 dependent cell line FDCP1. 6 well dishes were centrifuged for 1 hour at 1000×g, to increase the frequency of infection, followed by incubation at 37° C. for 24 hrs. The infected cells were washed four times to eradicate IL-3 from the media then plated into 96-well plates. Cells infected with retroviruses that contain an in-frame signal sequence and secreted mIL-3 were evidenced by live cells growing in each well after several days. After 2-3 weeks, genomic DNA was extracted from positive clones using standard methodologies.

### EXAMPLE 6

### cDNA Microarray Production

To amplify cDNAs using genomic DNA a nested PCR protocol was used. First round PCR primers were as follows: Forward 5′-CTGGTTTAGTGAACCGTCAGATC (SEQ ID NO:8) and Reverse 5′-CTCCTTGACAATAGAGCTGCAA (SEQ ID NO:9). The DNA was denaturated for 2 min at 94° C., and amplified by 35 cycles of 94° C. for 30 sec, 56° C. for 30 sec and 72° C. for 1 min, followed by a final extension of 7 min at 72° C. The first round PCR product was diluted 1:100 and 1 μl used as template for a second round of amplification to reduce the levels of genomic DNA in the final PCR product. Second round PCR primers were designed immediately adjacent to the plasmid insert site so the amplified cDNA product contains very little mIL-3 or vector sequence which may potentially interfere with hybridisation to the microarray chip. The primer sequences were as follows: Forward 5′-TAGCGCTACCGGACTCAGAT (SEQ ID NO:10) and Reverse 5′-CGGCCACTGATTGAAGCTT (SEQ ID NO:11). PCR conditions were the same as for first round PCR. Products were visualized by TBE agarose gel (1.% w/v) electrophoresis at 6V/cm for 60 min to ensure successful amplification had taken place.

PCR products were purified using the ArrayIt vacuum manifold system (TeleChem International, Sunnyvale, Calif.) and resuspended in 20 μL of 1× spotting solution (TeleChem) at a concentration of approximately 0.5 mg/ml in 384 well plate format. 5 μL of the resuspended purified cDNA solution was transferred to 384 well uniplates (Whatman Inc, Clifton, USA). This cDNA was arrayed onto Super Amine Microarray Substrates (TeleChem) using a Chip Writer Pro robotic arrayer (BioRad) fitted with 32 Stealth SMP-03 quill tipped microarray pins (TeleChem). The distance between adjacent cDNA spots was 200 μM. Each pin drew 0.25 μL of cDNA and deposited approximately 0.6 nL on each slide. Humidity was maintained between 55-65% during printing. Spotted DNAs were allowed to dry overnight, after which the slides were washed and blocked as recommended by the manufacturer (TeleChem).

### EXAMPLE 7

### cDNA Microarray Hybridization

Fluorescently labeled cDNA was prepared from 20 μg of total RNA using an indirect labelling method (Superscript Indirect cDNA Labelling System, Invitrogen) as per the manufacturers instructions. cDNA synthesis was performed in a 40 μL reaction containing 5 μg oligo-dT primer, 400U Superscript III (Invitrogen), 1× first strand buffer, 0.01M DTT, 0.5 mM of each dATP, dCTP and dGTP, 0.150 mM dTTP (Amersham, Buckinghamshire, UK) and 0.2 mM aminoallyl-dUTP (Sigma, St. Louis, Mo.). Synthesis was conducted in a GeneAmp PCR System 9700 (PE Applied Systems) at 46° C. for 2 hours. The reaction was stopped by addition of 5 μl of 0.5M EDTA and RNA was hydrolyzed by addition of 20 μl of 1M NaOH at 70° C. for 20 minutes. The reaction was neutralized with 25 μl of 1M HCl and the cDNA was purified using SNAP purification kits according to manufacturer's instructions (Invitrogen) and eluted in nuclease-free water. The cDNA was concentrated by ethanol precipitation and the cDNA pellet was resuspended in 5 μl coupling buffer. Cy3 or Cy5 monofunctional NHS ester reactive dyes (Amersham) were dissolved in 5 μl of DMSO and added to the cDNA. The coupling reaction was conducted in the dark for 1 hour.

Dye-coupled cDNA was purified using SNAP DNA purification columns (Invitrogen), combined and added to 10 μg of mouse Cot1 DNA (Invitrogen). The cDNAs were again concentrated with Microcon 30 spin columns (Millipore). The cDNA was hybridized in a 50 μL volume containing the labeled cDNA, 50% v/v formamide, 5×SSC, 8 μg PolydA, 2.5×Denhardt's solution, 4 μg yeast tRNA and 0.1% SDS. The cDNA was then denatured at 98° C. for 2 min and maintained at 60° C. until required. The hybridisation solution was mounted onto an array slide under a Lifterslip (Erie Glass) and hybridisation was conducted in a humid hybridisation chamber, in a hybridisation oven, at 42° C. for 16 hours. Following hybridisation the array slides were removed from their chamber and washed for 2 min in each of a 0.5×SSC and 0.1% w/v SDS, 0.5×SSC and 0.01% w/v SDS, 0.6×SSC and 0.06% w/v SDS solution. The array slides were dried in a centrifuge for 1 min at 500×g.

Fluorescent images of the microarrays were acquired using a GenePix 4000B scanner (Axon Instruments, Union City Calif., USA) and the images were analyzed using GenePix Pro 5.1 (Axon Instruments). Slides were scanned for both Cy3 and Cy5 signal at a 10 μM pixel resolution. Laser intensity and amplification of the photomultiplier tubes were adjusted to ensure approximately equal overall signal intensity for both Cy3 and Cy5. False colour images were generated for each dye and combined to provide a representation of the relative Cy3 and Cy5 intensities. Individual cDNA spots were flagged if spot size was too small, if the overall signal intensity was too low, or if the Cy3 and Cy5 signal intensities within the spots were not linearly related. GenePix Pro allows for the ‘flagging’ of bad elements (defined by present GenePix Pro parameters as feature signal intensity; feature background; element morphology; elements size and the percentage of pixels greater than feature background) that were then excluded from further analysis.

Median Cy3 and Cy5 signal intensities for each cDNA spot were imported from Genepix Pro files and data transformation conducted using Acuity (Version 4.0, Axon Instruments). Signal intensities for each feature were corrected for local background and features that failed to meet quality criteria (e.g. low expression values, poor feature morphology; small feature size or small percentage of pixels greater than feature background) were omitted. The ratio of Cy3 to Cy5 was calculated and the data logarithmically transformed (base2). Signal intensity was normalized to the mean intensity of all respective signal intensities, providing a relative measure of gene expression for each element on the microarray slide.

The expression of genes encoding SST positive cDNAs in muscle tissue of lean, normoglycaemic, normoinsulinaemic (NGT), obese normoglycaemic, hyperinsulinaemic (IGT) or obese type 2 diabetic (T2D) P. obesus obesity and type 2 diabetes was determined using microarray analysis. Differential gene expression as measured by microarray was assessed using the independent samples t-test algorithm in the Acuity software. Genes were considered to be differentially expressed if the significance of the t-test was p<0.05. A number of differentially expressed genes were identified and their expression was confirmed by real time PCR.

### EXAMPLE 8

### CXS-740

Sequence of Psammomys obesus CXS-740

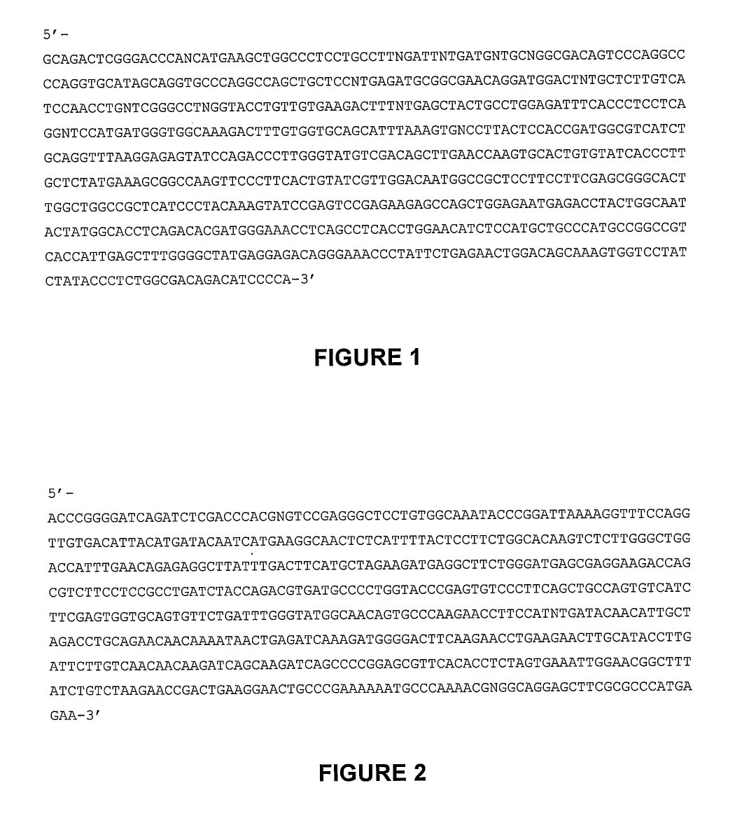

The nucleotide sequence identified shown in FIG. 1 (SEQ ID NO:1).

### CXS-740 Sequence Homology

Comparison of the P. obesus CXS-740 sequence to GenBank revealed it was homologous to the Sushi Domain Containing 2 (Susd2) gene. Alternate names for this gene are BK65A6.2 and Sushi domain (SCR repeat) containing.

### CXS-740 Bioinformatics

Susd2 is an 822 amino acid protein, has a signal peptide, and is thought to be a transmembrane protein with conserved Sushi domains. Molecular function of this protein is largely unknown, however it is suggested that Sushi domains may be implicated in signal transduction complexes. Susd2 also contains an adhesion associated domain, Somatomedin B-like domains, and a von Willebrand factor (vWF) type D domain. It is also predicted to have 2 coiled coil domains. The transmembrane domain is at the C terminal end at amino acids 786-808. Susd2 is located on chromosome 22 (22q11-q12).

SUSHI Domain: Present in a wide variety of complement and adhesion proteins, sushi domains are also known as complement control protein modules or short consensus repeats. Some members of the blood antigen group have this domain, with some members, such as CD21, also being part of signal transduction complexes. Sushi domains are known to be involved in many recognition processes, including the binding of several complement factors to fragments C3b and C4b.

Somatomedin B domain: A serum factor of unknown function, it is a cysteine-rich 45 residue peptide which is proteolytically derived from the N-terminal extremity of the cell-substrate adhesion protein vitronectin.

AMOP Domain: Molecular function unknown, but may have a role in cell adhesion. It is an extracellular domain and contains a number of cysteines that probably form disulphide bridges.

von Wildebrand Factor, Type D: The vWF domain is found in various plasma proteins: complement factors B, C2, CR3 and CR4; the integrins (I-domains); collagen types VI, VII, XII and XIV; and other extracellular proteins. The majority are extracellular proteins, however the most ancient ones present in all eukaryotes are all intracellular proteins involved in functions such as transcription, DNA repair, ribosomal and membrane transport and the proteasome. A common feature appears to be involvement in multiprotein complexes. Proteins that incorporate vWF domains participate in numerous biological events (e.g. cell adhesion, migration, homing, pattern formation, and signal transduction), involving interaction with a large array of ligands.

CXS-740 Gene Expression as Measured by SYBR Green Real Time PCR in P. obesus Skeletal Muscle

CXS-740 gene expression in the red gastrocnemius muscle of lean, normoglycaemic, normoinsulinaemic (NGT), obese normoglycaemic, hyperinsulinaemic (IGT) or obese type 2 diabetic (T2D) P. obesus, in the fed and fasted state, was analyzed by real time PCR. CXS-740 gene expression was significantly higher in fasted T2D than fasted NGT animals (p=0.001). In the fasted state, CXS-740 gene expression was significantly correlated with body weight (p=0.018) and plasma insulin (p=0.004). The results are shown in Table 4.

TABLE 4
CXS-740 gene expression in P. obesus skeletal muscle

| Group | Relative Gene Expression (±SEM) |
|---|---|
| NGT fed | 11.52 ± 2.96 |
| NGT fasted | 7.35 ± 2.03 |
| IGT fed | 11.52 ± 0.72 |
| IGT fasted | 8.75 ± 0.81 |
| T2D fed | 8.04 ± 1.06 |
| T2D fasted | 15.76 ± 1.34 |

These data show that elevated production of CXS-740 in skeletal muscle was associated with obesity and type 2 diabetes in this animal model.

### CXS-740 Gene Expression in Different Tissues as Measured by SYBR Green Real Time PCR

The CXS-740 gene was found to be expressed in all tissues examined in P. obesus, but was highest in the kidney, lung and skeletal muscle. The results are shown in Table 5.

TABLE 5
CXS-740 Tissue Distribution

| Tissue | Relative Gene Expression |
|---|---|
| Hypothalamus | 1.00 |
| Cortex | 1.75 |
| Cerebellum | 1.42 |
| Hippocampus | 0.51 |
| Midbrain | 0.62 |
| Brainstem | 1.88 |
| Liver | 0.14 |
| Red Gastrocnemius Muscle | 3.03 |
| White Gastrocnemius Muscle | 2.65 |
| Plantaris | 2.84 |
| EDL | 2.53 |
| Soleus muscle | 2.01 |
| Subscapular Fat | 0.44 |
| Intramuscular Fat | 0.40 |
| Epididymal Fat | 0.53 |
| Mesenteric Fat | 1.32 |
| Peri-renal Fat | 0.45 |
| Heart | 1.70 |
| Stomach | 0.59 |
| Kidney | 7.78 |
| Spleen | 1.20 |
| Adrenal | 0.58 |
| Large Intestine | 0.38 |
| Small Intestine | 0.28 |
| Lung | 3.77 |
| Testes | 1.56 |
| Ovary | 1.31 |

CXS-740 Gene Expression in L6 Muscle Cells Treated with Insulin as Measured by SYBR Green Real Time PCR

L6 cells were treated with 0, 0.1 nM, 1 nM, 10 nM, 100 nM or 1000 nM insulin for 6 hrs. Real time PCR showed that CXS-740 gene expression was significantly increased following treatment with 10 nM (p=0.014), 100 nM (p=0.04) and 1000 nM (p=0.004) insulin. The results are shown in Table 6.

TABLE 6
CXS-740 gene expression in L6 cells treated with insulin for 6 hrs

| | |
|---|---|
| Group | Relative Gene Expression (±SEM) |

| | | |
|---|---|---|
| 0 | nM Insulin | 103.57 ± 15.05 |
| 0.1 | nM Insulin | 142.27 ± 41.32 |
| 1 | nM Insulin | 103.28 ± 9.97 |
| 10 | nM Insulin | 231.34 ± 40.87 |
| 100 | nM Insulin | 208.14 ± 34.88 |
| 1000 | nM Insulin | 249.99 ± 31.78 |

L6 cells were also treated with 0, 0.1 nM, 1 nM, 10 nM, 100 nM or 1000 nM insulin for 1 hr. As seen with the 6 hr treatment, insulin treatment increased CXS-740 gene expression and this was significant with 0.1 nM (p=0.041) and 10 nM (p=0.044) insulin. The results are shown in Table 7.

TABLE 7
CXS-740 gene expression in L6 cells treated with insulin for 1 hr

| | |
|---|---|
| Group | Relative Gene Expression (±SEM) |

| | | |
|---|---|---|
| 0 | nM Insulin | 1.03 ± 0.11 |
| 0.1 | nM Insulin | 1.79 ± 0.42 |
| 1 | nM Insulin | 1.54 ± 0.22 |
| 10 | nM Insulin | 1.74 ± 0.32 |
| 100 | nM Insulin | 1.26 ± 0.11 |
| 1000 | nM Insulin | 1.71 ± 0.18 |

### EXAMPLE 9

### CXS-741

Sequence of Psammomys obesus CXS-741

The nucleotide sequence identified is shown in FIG. 2 (SEQ ID NO:2).

### CXS-741 Sequence Homology

Comparison of the P. obesus CXS-741 sequence to GenBank revealed it was the homologue of the Decorin (Dcn) gene. Alternate names for this gene and its product are DSPG2, PG40, PGII, PGE2, SLRR1B, bone proteoglycan II, dermatan sulphate proteoglycans II, proteoglycan core protein, small leucine-rich protein 1B, decorin precursor (bone proteoglycan II) (PG-S2) (PG40).

### CXS-741 Bioinformatics

Decorin is a 359 amino acid protein with a signal peptide. The human decorin gene is located on chromosome 12, spans more than 38 kb, contains 8 exons and very large introns (Danielson et al, Genomics 15 (1):146-160, 1993). There are multiple alternatively spliced transcript variants known for this gene.

Decorin is a small cellular or pericellular matrix proteoglycan that is closely related in structure to biglycan protein. The encoded protein and biglycan are thought to be the result of a gene duplication. This protein is a component of connective tissue, binds to Type I collagen fibrils, interacts with growth factors and plays a role in matrix assembly (Danielson et al., 1993 Supra). It contains one attached glycosaminoglycan chain. This protein is capable of suppressing the growth of various tumour cell lines. This gene is a candidate gene for Marfan syndrome.

Decorin is a potent regulator of TGF-β activity (Olguin et al, Dev Biol 259 (2):209-224, 2003), playing a functional role in regulating TGF-β signalling through decorin-induced Ca2+ signalling and inhibition of the Smad pathway (Abdel-Wahab et al, Biochem J 362 (3):643-649, 2002). Decorin regulates TGF-β1 activity and expression. It stops TGF binding to the TGF receptor.

Schonherr et al, J. Biol. Chem.: M500451200, 2005, have shown decorin to be involved in insulin-like growth factor-I (IGF-I) signaling (Schonherr et al, 2005 Supra). It binds to the IGF-I receptor on endothelial cells, with an affinity comparable to IGF-I, and activates its tyrosine kinase activity. Decorin addition causes IGF-I receptor phosphorylation and activation, which is followed by receptor downregulation. The IGF-IR is a ligand activated tyrosine protein kinase highly homologous to the insulin receptor. Decorin can also interact with IGF-I itself, regulating IGF signaling.

The decorin protein contains leucine-rich repeats (LRRs). Proteins containing LRRs are associated with widely different functions, a common property involves protein-protein interaction and cellular adhesion. Other functions of LRR-containing proteins include, for example, binding to enzymes and vascular repair. LRRs form elongated non-globular structures and are often flanked by cysteine rich domains. Decorin contains a cysteine rich domain N-terminal to the LRRs.

Decorin belongs to Class I Small Leucine-rich Proteoglycans (SLRPs), containing a pro-peptide. Ameye and Young (2002) reviewed research on SLRP-deficient mice, stating that decorin-deficient mice introduce tissue-specific variations in collagen fibril size, causing mice to have a fragile skin with reduced tensile strength and a thin dermis, mimicking defects observed in Ehlers-Danlos syndrome (Ameye and Young, Glycobiology 12 (9):107R-116R, 1999). Diseases such as osteoporosis, osteoarthritis, muscular dystrophy, Ehlers-Danlos syndrome and corneal diseases are also developed by SLRP-deficient mice (Ameye and Young, 1999 Supra).

In vitro studies with 313-L1 cells have highlighted the major role of proteoglycans in adipocyte growth and differentiation (Calvo et al, J. Biol. Chem. 266 (17):11237-11244, 1991). Musil et al (J Cell Biol 114 (4):821-826, 1991) have investigated the accumulation of proteoglycans during the 313-L1 differentiation process, with mature adipocytes accumulating 50-70% less decorin compared to undifferentiated fibroblasts (Musil et al, 1991 Supra). Musil et al, 1991 Supra hypothesise that once differentiation is complete, cells only produce enough extracellular matrix components to maintain good cell-substrate and cell-cell adhesion.

Knoll et al, Physiol Genomics, 2005, experimented with tissue-specific molecular alterations in diabetes, comparing four important diabetic target tissues in rats with two weeks of streptozotocin-induced diabetes. They found that decorin was up-regulated in several tissues (Knoll 2005 Supra). Schaefer et al (Faseb J. 15 (3):559-561, 2001) showed decorin to be overexpressed in renal tissue from patients with diabetic nephropathy (Schaefer et al, 2001 Supra) Mogyorosi and Ziyadeh, (Nephrol Dial Transplant 14 (5):1078-1081, 1999) conclude that decorin abundance is increased in both in vitro and in vivo models of diabetic kidney disease (Mogyorosi et al, 1999 Supra).

Olsson et al (Diabetes 48 (3):616-622, 1999) researched the effect of nonesterified fatty acids (NEFA) on the expression of genes coding for extracellular matrix proteoglycans and found that in smooth muscle cells, alterations of cell morphology and an increase in decorin expression could be seen. In addition, the NEFAs increased the size of the glycosaminoglycan moiety of decorin itself (Olsson et al, 1999 Supra).

Decorin is expressed by sprouting endothelial cells (ECs) during inflammation-induced angiogenesis in vivo and by human endothelial cells co-cultured with fibroblasts in a collagen lattice. Interleukin (IL)-6 and IL-10, two cytokines released during inflammation, induce decorin mRNA in endothelial cells, but interaction with fibrillar collagen is essential for its translation (Strazynski et al, 2004).

CXS-741 Gene Expression as Measured by SYBR Green Real Time PCR in P. obesus Skeletal Muscle

CXS-741 gene expression in the red gastrocnemius muscle of NGT, IGT and T2D P. obesus, in the fed and fasted state, was analyzed by real time PCR. CXS-741 gene expression was significantly higher in muscle of fasted T2D animals compared with fasted NGT (p=0.001) and IGT (p=0.008) animals. Gene expression was positively correlated with plasma insulin concentration (p=0.005) and body weight (p=0.029) in the fasted state. When animals were in the fed state, CXS-741 gene expression was higher in muscle of IGT compared with NGT animals (p=0.029). The results are shown in Table 8.

Therefore, increased production of CXS-741 (decorin) in skeletal muscle was associated with obesity, insulin resistance and type 2 diabetes in this animal model.

TABLE 8
CXS-741 gene expression in P. obesus skeletal muscle

| Group | Relative Gene Expression (±SEM) |
|---|---|
| NGT fed | 12.52 ± 2.96 |
| NGT fasted | 3.55 ± 0.49 |
| IGT fed | 33.07 ± 8.97 |
| IGT fasted | 6.36 ± 2.07 |
| T2D fed | 33.86 ± 8.86 |
| T2D fasted | 34.47 ± 10.54 |

### CXS-741 Gene Expression in Different Tissues as Measured by SYBR Green Real Time PCR

The CXS-741 gene was found to be expressed in all tissues examined in P. obesus, but was highest in the adipose tissue, heart and lung. The results are shown in Table 9.

TABLE 9
CXS-741 Tissue Distribution

| Tissue | Relative Gene Expression |
|---|---|
| Hypothalamus | 1.00 |
| Cortex | 0.46 |
| Cerebellum | 0.48 |
| Hippocampus | 0.23 |
| Midbrain | 0.17 |
| Brainstem | 0.81 |
| Liver | 0.64 |
| Red Gastrocnemius Muscle | 1.64 |
| White Gastrocnemius Muscle | 0.58 |
| Plantaris | 0.36 |
| EDL | 0.18 |
| Soleus muscle | 0.85 |
| Subscapular Fat | 20.89 |
| Intramuscular Fat | 7.46 |
| Epididymal Fat | 13.18 |
| Mesenteric Fat | 43.87 |
| Peri-renal Fat | 7.52 |
| Heart | 12.77 |
| Stomach | 3.69 |
| Kidney | 0.13 |
| Spleen | 0.61 |
| Adrenal | 0.83 |
| Large Intestine | 0.20 |
| Small Intestine | 0.39 |
| Lung | 8.78 |
| Testes | 0.52 |
| Ovary | 5.94 |

CXS-741 Gene Expression as Measured by SYBR Green Real Time PCR in P. obesus Mesenteric Fat

CXS-741 gene expression in the mesenteric fat of NGT, IGT and T2D P. obesus, in the fed and fasted state, was analyzed by real time PCR. In the fasted state, CXS-741 gene expression was significantly higher in IGT and T2D compared with NGT animals (p=0.029 and p=0.014, respectively). Furthermore, CXS-741 gene expression was positively correlated with plasma insulin concentration (p=0.006) and body weight (p=0.017). In the fed state, there was also a trend for increased CXS-741 gene expression in IGT and T2D animals. The results are shown in Table 10.

Therefore, as in skeletal muscle, increased production of CXS-741 (decorin) in adipose tissue was associated with obesity, insulin resistance and type 2 diabetes in this animal model.

TABLE 10
CXS-741 gene expression in P. obesus mesenteric fat

| Group | Relative Gene Expression (±SEM) |
|---|---|
| NGT fed | 174.39 ± 53.66 |
| NGT fasted | 77.90 ± 25.67 |
| IGT fed | 401.94 ± 99.28 |
| IGT fasted | 257.59 ± 80.87 |
| T2D fed | 383.26 ± 156.15 |
| T2D fasted | 269.22 ± 64.56 |

CXS-741 Gene Expression in 3T3-L1 Adipocytes Treated with Insulin as Measured by SYBR Green Real Time PCR

Differentiated 3T3-L1 adipocyte cells were treated with 0, 0.1 nM, 1 nM, 10 nM, 100 nM or 1000 nM insulin for 6 hrs. Real time PCR showed that CXS-741 gene expression was significantly increased following treatment with 1000 nM insulin (p=0.049).

TABLE 11
CSX-741 gene expression in 3T3-L1 adipocytes
treated with insulin for 6 hrs

| | |
|---|---|
| Group | Relative Gene Expression (±SEM) |

| | | |
|---|---|---|
| 0 | nM Insulin | 1.02 ± 0.10 |
| 0.1 | nM Insulin | 1.20 ± 0.17 |
| 1 | nM Insulin | 1.42 ± 0.06 |
| 10 | nM Insulin | 1.08 ± 0.11 |
| 100 | nM Insulin | 1.29 ± 0.14 |
| 1000 | nM Insulin | 1.47 ± 0.24 |

### CXS-741 Gene Expression in 3T3-L1 Adipocytes During Differentiation as Measured by SYBR Green Real Time PCR

3T3-L1 adipocyte cells were cultured in high glucose DMEM (25 mM), supplemented with 10% fetal bovine serum, 50 units/ml penicillin and 50 μg/ml streptomycin. Two days after the cells reached confluence, differentiation of fibroblasts into adipocytes was initiated by the addition of high glucose DMEM, 10% fetal bovine serum, 50 units/ml penicillin and 50 μg/ml streptomycin, 0.5 mM 1-methyl-3-isobutylxanthine (IBMX), 2.5 μM dexamthasone and 0.166 U/ml insulin for three days. The medium was then aspirated and replaced glucose DMEM, 10% fetal bovine serum, 50 units/ml penicillin and 50 μg/ml streptomycin and 0.166 U/ml insulin for two days. Following these treatments, the cells were cultured in glucose DMEM, 10% fetal bovine serum, 50 units/ml penicillin and 50 μg/ml streptomycin. Real time PCR showed that CXS-741 gene expression was significantly increased following differentiation compared to undifferentiated fibroblasts on day 2 (p=0.016), day 5 (p=0.006), day 6 (p=0.001) and day 7 (p=0.012) post differentiation. This data clearly shows that mature adipocytes express high levels of CXS-741 and suggest that the expression of CXS-742 observed in adipose tissue is predominantly within adipocytes.

TABLE 12
CSX-741 gene expression in 3T3-L1 adipocytes
undergoing differentiation

| Culture Conditions | Relative Gene Expression (±SEM) |
|---|---|
| Day 1: 2 days post 100% confluence, | 10.23 ± 0.97 |
| normal growth media | |
| Day 2: differentiation with the | 55.37 ± 7.68 |
| addition of IBMX, dexamethasone, | |
| insulin | |
| Day 3: differentiation with the | 16.86 ± 2.46 |
| addition of IBMX, dexamethasone, | |
| insulin | |
| Day 4: differentiation with the | 64.67 ± 16.68 |
| addition of IBMX, dexamethasone, | |
| insulin | |
| Day 5: differentiation with the | 83.32 ± 9.90 |
| addition of insulin | |
| Day 6: differentiation with the | 61.65 ± 5.05 |
| addition of insulin | |
| Day 7: normal growth media | 49.58 ± 6.34 |

### CXS-741 Gene Expression in C2C12 Muscle Cells During Differentiation as Measured by SYBR Green Real Time PCR

C2C12 muscle cells were cultured in high glucose DMEM (25 mM), supplemented with 10% fetal bovine serum. At day 3, when the cells has become 100% confluent, differentiation was initiated by the addition of high glucose DMEM supplemented with 2% fetal bovine serum and cells were continued to be cultured in this media for five days. Real time PCR showed that CXS-741 gene expression was significantly increased at days 2, 3, 5, 6, 8 of differentiation compared to undifferentiated cells at day 1 (p<0.05).

TABLE 13
CSX-741 gene expression in C2C12 muscle
cells undergoing differentiation

| Culture Conditions | Relative Gene Expression (±SEM) |
|---|---|
| Day 1: 60% confluent | 4.79 ± 1.03 |
| Day 2: 80% confluent | 22.16 ± 1.23 |
| Day 3: 100% confluent, | 96.63 ± 3.95 |
| differentiation induced | |
| with the addition of high | |
| glucose DMEM supplemented | |
| with 2% fetal bovine serum | |
| Day 4: high glucose DMEM | 87.79 ± 12.74 |
| supplemented with 2% fetal | |
| bovine serum | |
| Day 5: high glucose DMEM | 70.63 ± 5.40 |
| supplemented with 2% fetal | |
| bovine serum | |
| Day 6: high glucose DMEM | 139.45 ± 8.19 |
| supplemented with 2% fetal | |
| bovine serum | |
| Day 7: high glucose DMEM | 167.95 ± (n = 1) |
| supplemented with 2% fetal | |
| bovine serum | |
| Day 8: high glucose DMEM | 145.38 ± 8.52 |
| supplemented with 2% fetal | |
| bovine serum | |

CXS-741 Gene Expression as Measured by SYBR Green Real Time PCR in P. obesus Subscapular Fat

CXS-741 gene expression in the subscapular fat of fed NGT, IGT and T2D P. obesus, was analyzed by real time PCR. CXS-741 gene expression was significantly higher in IGT and T2D than NGT animals (p=0.024 and p=0.020, respectively). Furthermore, CXS-741 gene expression was positively correlated with plasma insulin concentration (p=0.004), body weight (p=0.001), glucose concentration (p=0.017) and percent body fat (p=0.002).

TABLE 14
CSX-741 gene expression in P. obesus subscapular fat

| Group | Relative Gene Expression (±SEM) |
|---|---|
| NGT fed | 117.81 ± 25.16 |
| IGT fed | 677.01 ± 159.77 |
| T2D fed | 1062.63 ± 227.79 |

CXS-741 Gene Expression as Measured by Western Blot in P. obesus Subscapular Fat Protein

Two NGT, three IGT and three T2D subscapular fat protein samples (30 μg/sample) from fed P. obesus were analyzed for CXS-741 expression by Western blot with anti-CXS-741 antibodies. The CXS-741 antibody detected a band at 45 kDa in these experiments. CXS-741 protein expression was higher in IGT and T2D animals compared to NGT, which is consistent with the mRNA expression profile observed previously in P. obesus mesenteric and subscapular adipose tissues.

TABLE 15
Ratio change of CXS-741 protein expression
in P. obesus subscapular fat protein

| Group | Ratio Change |
|---|---|
| NGT | 1 |
| IGT | 1.61 |
| T2D | 1.86 |

CXS-741 Gene Expression as Measured by SYBR Green RT-PCR in P. obesus Visceral and Subcutaneous Adipose Tissue

CXS-741 gene expression in fed NGT, IGT and T2D P. obesus visceral and subcutaneous adipose tissue was analyzed by real time PCR. CXS-741 gene expression was significantly higher overall in visceral compared to subcutaneous adipose tissue (p=0.002). CXS-741 gene expression was significantly higher in IGT visceral adipose tissue compared to IGT subcutaneous adipose tissue (p=0.05) and T2D visceral adipose tissue compared to T2D subcutaneous adipose tissue (p=0.005). This data identifies CXS-741 as a new adipocytokine that is preferentially produced by abdominal visceral fat, the accumulation of which has been linked to increased risk of insulin resistance and type 2 diabetes. Moreover, the greater expression of CXS-741 in visceral adipose tissue of IGT and T2D compared with nGT P. obesus raises the possibility that CXS-741 may contribute to the development of insulin resistance and dysregulation of glucose homeostasis associated with type 2 diabetes.

TABLE 16
CSX-741 gene expression in P. obesus
visceral and subcutaneous adipose tissue

| Group | Relative Gene Expression (±SEM) |
|---|---|
| NGT visceral | 5.66 ± 1.41 |
| NGT subcutaneous | 34.40 ± 9.35 |
| IGT visceral | 30.77 ± 7.66 |
| IGT subcutaneous | 80.29 ± 11.52 |
| T2D visceral | 45.03 ± 9.85 |
| T2D subcutaneous | 115.91 ± 34.46 |

### CXS-741 Gene Expression as Measured by ELISA in Human Plasma Samples

A CXS-741 ELISA was developed to measure CXS-741 levels in human plasma from 145 NGT and 142 T2D human subjects. Plasma CXS-741 measured by ELISA was found to be significantly elevated by 14% in T2D subjects (p=0.043). Furthermore, in the male subjects plasma CXS-741 levels correlated with waist circumference (p=0.007), and both fasting and 2-hour glucose levels in an OGTT (p=0.024 and p=0.001, respectively). These findings suggest that circulating plasma CXS-741 may contribute to insulin resistance and therapeutic agents designed to alter CXS-741 levels or that modulate CXS-741 activity may be useful therapies in the treatment of type 2 diabetes and related disorders.

In addition, elevated circulating levels of CXS-741 can also be used to predict the onset of diabetes and conditions associated therewith, such as blindness, nephropathy and/or cardiovascular disease and/or inflammation.

TABLE 17
Plasma CXS-741 in NGT and T2D human plasma samples

| Group | Relative Gene Expression (±SEM) |
|---|---|
| NGT | 1.8 ± 0.09 |
| T2D | 2.06 ± 0.13 |

### EXAMPLE 10

### CXS-742

Sequence of Psammomys obesus CXS-742

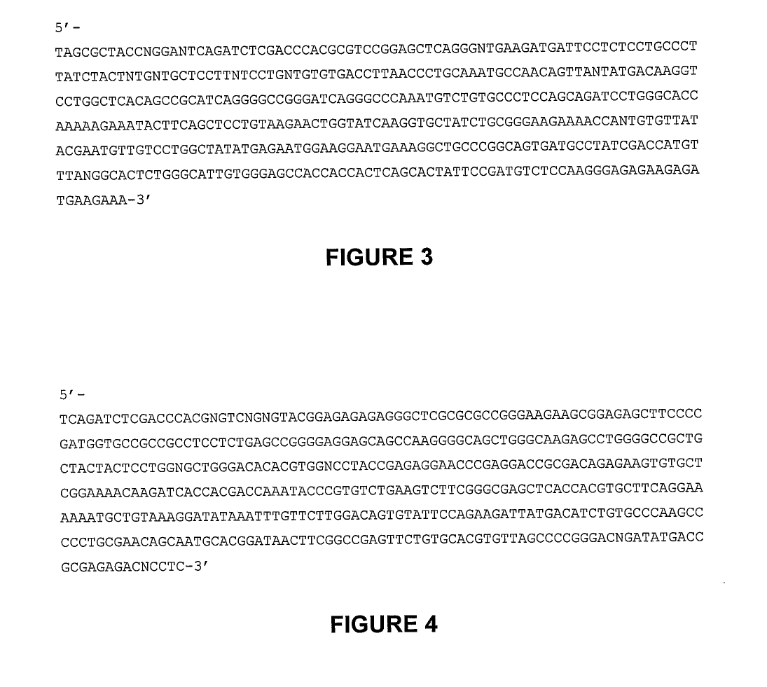

The nucleotide sequence identified is shown in FIG. 3 (SEQ ID NO:3).

### CXS-742 Sequence Homology

Analysis of the P. obesus CXS-742 sequence revealed that it was homologous to the periostin gene. Alternate names for this gene and its gene product are OSF-2, periostin precursor (PN), POSTN, osteoblast specific factor 2 (fascilclin I-like), periostin osteoblast specific factor.

### CXS-742 Literature Review and Bioinformatics

Periostin is an 836 amino acid protein with a signal peptide. It is mapped to chromosome 13 (13q13.3).

Periostin is a secreted protein and induces cell attachment/cell adhesion. It is highly expressed in early osteoblastic cells in vivo and in periosteum and periodontal ligament tissues in vivo (Oshima et al, J Cell Biochem 96 (4):792-804, 2002). Periostin is involved in cell adhesion and spreading in vitro (Oshima et al, 2002 Supra). Bao et al (Cancer Cell 5 (4):329-339, 2004) found that periostin was overexpressed in >80% of human colon cancers, with highest expression in metastatic tumours, activating the Akt/PKB signalling pathway though alpha(v)beta(3) integrins to increase cellular survival[17]. Overexpression of Twist, an important transcription factor for cell type determination and differentiation, resulted in up-regulation of periostin expression (Oshima et al, 2002 Supra).

Periostin contains the following domains:

EMI Domain: Amino acids 40-94; The EMI domain, first named after its presence in proteins of the EMILIN family, is a small cysteine-rich module of ˜75 amino acids. The EMI domain is most often found at the N-terminus of metazoan extracellular proteins that are forming or are compatible with multimer formation. It is found in association with other domains, such as C1q, laminin-type EGF-like, collagen-like, FN3, WAP, ZP or FAS1. It has been suggested that the EMI domain could be a protein-protein interaction module, as the EMI domain of EMILIN-1 was found to interact with the C1q domain of EMILIN-2. Extracellular matrix glycoproteins contain this domain.

4 FAS1/BlgH3 domains: From amino acids 97-230, 234-365, 368-492, 496-628; The FAS1 or BIgH3 domain is an extracellular cell adhesion module of about 140 amino acid residues. Most FAS1 domain containing proteins are GPI anchored and contain two or four copies of the domain. FAS1 domains of BIgH3 protein mediate cell adhesion through an interaction with alpha3/beta1 integrin.

CXS-742 Gene Expression as Measured by SYBR Green Real Time PCR in P. obesus Skeletal Muscle

CXS-742 gene expression in the red gastrocnemius muscle of NGT, IGT and T2D P. obesus, in the fed and fasted state, was analyzed by real time PCR. CXS-742 gene expression in skeletal muscle tended to be increased in IGT and T2D animals in both the fed and fasted states, and was significantly positively correlated with blood glucose concentration (p=0.049) in fasted animals. The results are shown in Table 18.

TABLE 18
CXS-742 gene expression in P. obesus skeletal muscle

| Group | Relative Gene Expression (±SEM) |
|---|---|
| NGT fed | 11.43 ± 2.70 |
| NGT fasted | 4.40 ± 1.68 |
| IGT fed | 23.52 ± 5.84 |
| IGT fasted | 11.65 ± 4.47 |
| T2D fed | 20.79 ± 1.54 |
| T2D fasted | 17.17 ± 5.73 |

CXS-742 Gene Expression as Measured by SYBR Green Real Time PCR in P. obesus Adipose Tissue

CXS-742 gene expression in the mesenteric fat of NGT, IGT and T2D P. obesus, in the fed and fasted state, was analyzed by real time PCR. Gene expression tended to be increased in fed and fasted IGT and T2D animals, a similar pattern to in the muscle, but did not reach statistical significance. In the fed state, gene expression positively correlated with body weight (p=0.032). The results are shown in Table 19.

Together with the gene expression data in skeletal muscle, these data suggest that increased expression of CXS-742 is associated with obesity and type 2 diabetes in this animal model.

TABLE 19
CXS-742 gene expression in P. obesus mesenteric fat

| Group | Relative Gene Expression (±SEM) |
|---|---|
| NGT fed | 148.57 ± 43.07 |
| NGT fasted | 148.05 ± 65.10 |
| IGT fed | 330.25 ± 56.84 |
| IGT fasted | 273.63 ± 67.29 |
| T2D fed | 310.25 ± 94.78 |
| T2D fasted | 261.06 ± 74.70 |

### CXS-742 Gene Expression in Different Tissues as Measured by SYBR Green Real Time PCR

The CXS-742 gene was found to be most highly expressed in adipose tissue in P. obesus (Table 20).

TABLE 20
CXS-742 Tissue Distribution

| Tissue | Relative Gene Expression |
|---|---|
| Hypothalamus | 1.00 |
| Cortex | 45.41 |
| Cerebellum | 4.00 |
| Hippocampus | 3.62 |
| Midbrain | 4.96 |
| Brainstem | 1.34 |
| Liver | 2.68 |
| Red Gastrocnemius Muscle | 9.25 |
| White Gastrocnemius Muscle | 3.77 |
| Plantaris | 0.86 |
| EDL | 1.01 |
| Soleus muscle | 8.22 |
| Subscapular Fat | 469.51 |
| Intramuscular Fat | 467.88 |
| Epididymal Fat | 741.86 |
| Mesenteric Fat | 586.10 |
| Peri-renal Fat | 1120.56 |
| Heart | 43.26 |
| Stomach | 51.27 |
| Kidney | 2.34 |
| Spleen | 5.17 |
| Adrenal | 23.67 |
| Large Intestine | 8.06 |
| Small Intestine | 13.00 |
| Lung | 267.80 |
| Testes | 0.38 |
| Ovary | 150.64 |

CXS-742 Gene Expression as Measured by SYBR Green Real Time PCR in P. obesus Subscapular Fat

CXS-742 gene expression in the subscapular fat of fed NGT, IGT and T2D P. obesus, was analyzed by real time PCR. CXS-742 gene expression was significantly higher in T2D compared to NGT animals (p=0.021). Furthermore, CXS-742 gene expression was positively correlated with body weight (p=0.003), and percent body fat (p=0.011). This data identifies CXS-742 as a new adipocytokine that more highly expressed in the adipose tissue of IGT and T2D compared with nGT P. obesus. This data raises the possibility that CXS-742 may contribute to the development of insulin resistance and dysregulation of glucose homeostasis associated with type 2 diabetes.

TABLE 21
CSX-742gene expression in P. obesus subscapular fat

| Group | Relative Gene Expression (±SEM) |
|---|---|
| NGT fed | 108.96 ± 14.41 |
| IGT fed | 247.36 ± 56.31 |
| T2D fed | 271.31 ± 40.98 |

### EXAMPLE 11

### CXS-743

The nucleotide sequence identified is shown in FIG. 4 (SEQ ID NO:4).

### CXS-743 Sequence Homology

Analysis of the P. obesus CXS-743 sequence revealed it was homologous to the Collagen and calcium binding EGF domain 1 (CCBE1) gene. Alternate names for this gene and its product are RIKEN cDNA 9430093N24 (9430093N24Rik), KIAA1983 and FLJ30681.

CXS-743 Bioinformatics

CCBE1 is a 406 amino acid protein with a signal peptide. The CCBE1 gene has been mapped to chromosome 18 (18q21.32). The function of this protein is unknown.

Bioinformatics predicts 2 coiled coil regions, and 1 PEST sequence, indicating this protein may be targeted for relatively rapid degradation. CCBE1 contains the following domains/motifs:

EGF1-like Domain: From amino acids 134-175, with 3 predicted disulphide bonds. A sequence of about thirty to forty amino-acid residues long found in the sequence of epidermal growth factor (EGF) has been shown to be present, in a more or less conserved form, in a large number of other, mostly animal proteins. EGF is a polypeptide of about 50 amino acids with three internal disulfide bridges. It first binds with high affinity to specific cell-surface receptors and then induces their dimerization, which is essential for activating the tyrosine kinase in the receptor cytoplasmic domain, initiating a signal transduction that results in DNA synthesis and cell proliferation. A common feature of all EGF-like domains is that they are found in the extracellular domain of membrane-bound proteins or in proteins known to be secreted.

Calcium-binding EGF-like domain: Amino acids 134-159; Many of the proteins that contain the above EGF-like domain require calcium for their biological function. A calcium-binding site has been found to be located at the N-terminus of some EGF-like domains. Calcium-binding may be crucial for numerous protein-protein interactions. Proteins that are known or that are predicted to contain calcium-binding EGF-like domains are: Calcium dependent serine proteinase, which degrades extracellular matrix proteins; Coagulation factors VII, IX, X; Neurogenic proteins; and Fibrillin 1 & 2.

Aspartic acid and Asparagine hydroxylation site: Amino acids 150-161; Post-translational hydroxylation of aspartic acid or asparagine to form erythro-beta-hydroxyaspartic acid or erythro-beta-hydroxyasparagine has been identified in a number of proteins with domains homologous to epidermal growth factor (EGF). Examples of such proteins are the blood coagulation protein factors VII, IX and X, proteins C, S, and Z, the LDL receptor, thrombomodulin, etc. Based on sequence comparisons of the EGF-homology region that contains hydroxylated Asp or Asn, a consensus sequence has been identified that seems to be required by the hydroxylase(s).

Collagen Triple Helix Repeat: Amino acids 246-333; Members of this family belong to the collagen superfamily, with the repeats forming a triple helix structure. Collagens are generally extracellular structural proteins involved in formation of connective tissue structure. Collagens are post-translationally modified by proline hydroxylase to form the hydroxyproline residues. Defective hydroxylation is the cause of scurvy. Some members of the collagen superfamily are not involved in connective tissue structure but share the same triple helical structure.

vWA_Matrilin domain. In cartilaginous plate, extracellular matrix molecules mediate cell-matrix and matrix-matrix interactions thereby providing tissue integrity.

CXS-743 Gene Expression as Measured by SYBR Green Real Time PCR in P. obesus Skeletal Muscle

CXS-743 gene expression in red gastrocnemius muscle of NGT, IGT and T2D P. obesus, in the fed and fasted state, was analyzed by real time PCR. CXS-743 gene expression was not statistically different between P. obesus groups, despite a strong trend for increased expression in T2D fasted animals. However, in the fasted state CXS-743 gene expression was significantly correlated with plasma insulin concentration (p=0.006) and body weight (p=0.020) (Table 22).

These data suggest that increased production of CXS-743 is associated with obesity and type 2 diabetes.

TABLE 22
CXS-743 gene expression in P. obesus skeletal muscle

| Group | Relative Gene Expression (±SEM) |
|---|---|
| NGT fed | 11.71 ± 2.48 |
| NGT fasted | 6.45 ± 0.76 |
| IGT fed | 15.26 ± 2.43 |
| IGT fasted | 8.58 ± 1.46 |
| T2D fed | 14.70 ± 1.70 |
| T2D fasted | 18.65 ± 3.69 |

CXS-743 Gene Expression as Measured by SYBR Green Real Time PCR in P. obesus liver

CXS-743 gene expression in the liver of NGT, IGT and T2D P. obesus, in the fed and fasted state, was analyzed by real time PCR. In the fed state, CXS-743 gene expression was significantly reduced in the liver of T2D animals (p=0.048), and there was a significant correlation with blood glucose levels (p=0.018) (Table 23).

TABLE 23
CXS-743 gene expression in P. obesus liver

| Group | Relative Gene Expression (±SEM) |
|---|---|
| NGT fed | 11.24 ± 1.88 |
| NGT fasted | 9.37 ± 1.56 |
| IGT fed | 13.06 ± 1.36 |
| IGT fasted | 9.24 ± 0.66 |
| T2D fed | 9.46 ± 2.06 |
| T2D fasted | 8.94 ± 0.39 |

CXS-743 Gene Expression as Measured by SYBR Green Real Time PCR in P. obesus Adipose Tissue

CXS-743 gene expression in the mesenteric fat of NGT, IGT and T2D P. obesus, in the fed and fasted state, was analyzed by real time PCR. CXS-743 gene expression positively correlated with plasma insulin concentration (p=0.048) in fasted animals (Table 24).

These data support the skeletal muscle data, and suggest that elevated production of CXS-743 in muscle and fat is associated with obesity, insulin resistance and type 2 diabetes.

TABLE 24
CXS-743 gene expression in P. obesus mesenteric fat

| Group | Relative Gene Expression (±SEM) |
|---|---|
| NGT fed | 135.48 ± 34.29 |
| NGT fasted | 122.08 ± 39.02 |
| IGT fed | 230.43 ± 42.49 |
| IGT fasted | 202.22 ± 41.98 |
| T2D fed | 218.49 ± 64.93 |
| T2D fasted | 169.93 ± 30.29 |

### CXS-743 Gene Expression in Different Tissues as Measured by SYBR Green Real Time PCR

The CXS-743 gene was found to be most highly expressed in the adrenal gland, cerebral cortex and adipose tissue of P. obesus (Table 25).

TABLE 25
CXS-743 Tissue Distribution

| Tissue | Relative Gene Expression |
|---|---|
| Hypothalamus | 1.00 |
| Cortex | 20.53 |
| Cerebellum | 0.51 |
| Hippocampus | 6.32 |
| Midbrain | 3.39 |
| Brainstem | 0.73 |
| Liver | 0.51 |
| Red Gastrocnemius Muscle | 0.65 |
| White Gastrocnemius Muscle | 0.22 |
| Plantaris | 0.20 |
| EDL | 0.23 |
| Soleus muscle | 0.28 |
| Subscapular Fat | 3.47 |
| Intramuscular Fat | 6.08 |
| Epididymal Fat | 8.22 |
| Mesenteric Fat | 16.00 |
| Peri-renal Fat | 7.86 |
| Heart | 10.85 |
| Stomach | 0.34 |
| Kidney | 5.54 |
| Spleen | 7.54 |
| Adrenal | 47.84 |
| Large Intestine | 9.13 |
| Small Intestine | 3.31 |
| Lung | 7.09 |
| Testes | 3.31 |
| Ovary | 14.07 |

### EXAMPLE 12

### CXS-744

Sequence of Psammomys obesus CXS-744

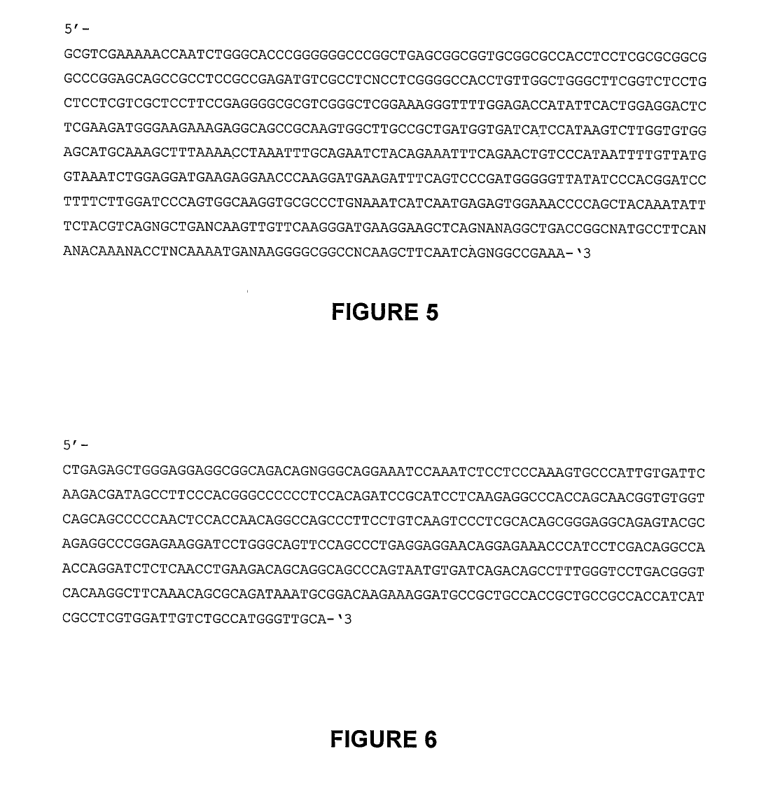

The nucleotide sequence identified is shown in FIG. 5 (SEQ ID NO:5)

### CXS-744 Sequence Homology

Analysis of the P. obesus CXS-744 sequence revealed it was homologous to the thioredoxin-like protein p19 (TLP19) gene. Alternate names for this gene and its product are Endoplasmic Reticulum Thioredoxin Superfamily Member, 18 kDa (ERP18), ERP19, thioredoxin-like p19 precursor, endoplasmic reticulum protein ERp19, similar to RIKEN cDNA 0610040B21 (LOC298370).

### CXS-744 Bioinformatics

The TLP19 protein is 172 amino acids long and has a signal peptide. Liu et al (Gene 315:71-78, 2003) mapped human TLP to chromosome 1p32.3 (Liu et al Supra).

Knocblach et al (Mol Cell Proteomics 2 (10):1104-1119, 2003) describe TLP19 to be an ER luminal protein, a member of the protein disulphide isomerase (PDI) family (Knocblach et al, 2003 Supra). Immunoblotting assays indicated it might also be secreted out of the cell through the trans-Golgi network (Lieu et al, 2003 Supra).

The TLP19 protein has a thioredoxin domain at amino acids 58-76. Thioredoxins are small proteins of approximately one hundred amino acid residues which participate in various redox reactions via the reversible oxidation of an active center disulfide bond. They exist in either a reduced form or an oxidized form, where the two cysteine residues are linked in an intramolecular disulfide bond. Alanen et al (J Biol Chem 278 (31):28912-28920, 2003) hypothesise that TLP19 is involved in disulfide bond formation and not reduction, and that it might possess significant peptide thiol-disulfide oxidase activity (Alanen et al, 2003 Supra).

CXS-744 Gene Expression as Measured by SYBR Green Real Time PCR in P. obesus Skeletal Muscle

CXS-744 gene expression in the red gastrocnemius muscle of NGT, IGT and T2D P. obesus, in the fed and fasted state, was analyzed by real time PCR. In the fed state, CXS-744 gene expression in muscle was strongly correlated with blood glucose levels (p<0.001).

TABLE 26
CXS-744 gene expression in P. obesus skeletal muscle

| Group | Relative Gene Expression (±SEM) |
|---|---|
| NGT fed | 10.88 ± 1.93 |
| NGT fasted | 7.64 ± 0.95 |
| IGT fed | 11.02 ± 0.66 |
| IGT fasted | 8.82 ± 1.67 |
| T2D fed | 7.43 ± 0.27 |
| T2D fasted | 8.36 ± 0.37 |

### CXS-744 Gene Expression in Different Tissues as Measured by SYBR Green Real Time PCR

The CXS-744 gene was found to be expressed in all tissues examined in P. obesus, with highest levels in the adrenal gland, testes and adipose tissue (Table 27).

TABLE 27
CXS-744 Tissue Distribution

| Tissue | Relative Gene Expression |
|---|---|
| Hypothalamus | 1.00 |
| Cortex | 0.87 |
| Cerebellum | 1.00 |
| Hippocampus | 0.72 |
| Midbrain | 1.02 |
| Brainstem | 1.00 |
| Liver | 0.57 |
| Red Gastrocnemius Muscle | 0.60 |
| White Gastrocnemius Muscle | 0.58 |
| Plantaris | 0.64 |
| EDL | 0.82 |
| Soleus muscle | 0.74 |
| Subscapular Fat | 1.29 |
| Intramuscular Fat | 1.63 |
| Epididymal Fat | 1.58 |
| Mesenteric Fat | 1.33 |
| Peri-renal Fat | 1.75 |
| Heart | 0.78 |
| Stomach | 1.05 |
| Kidney | 0.49 |
| Spleen | 0.38 |
| Adrenal | 6.32 |
| Large Intestine | 0.16 |
| Small Intestine | 0.36 |
| Lung | 1.47 |
| Testes | 2.17 |
| Ovary | 1.06 |

CXS-744 Gene Expression in L6 Muscle Cells Treated with Insulin as Measured by SYBR Green Real Time PCR

L6 cells were treated with 0, 0.1 nM, 1 nM, 10 nM, 100 nM or 1000 nM insulin for 6 hrs. Real time PCR showed that CXS-744 gene expression was significantly increased following treatment with 1 nM (p=0.012), 10 nM (p=0.003), 100 nM (p<0.001) and 1000 nM (p<0.001) insulin. The increase was dose-dependent. Cells treated with 100 nM and 1000 nM insulin had significantly higher gene expression than cells treated with 0.1 nM and 1 nM (p<0.001 for all). Cells treated with 100 nM and 1000 nM insulin had significantly higher CXS-744 gene expression than cells treated with 10 nM (p=0.005 and p=0.001, respectively) (Table 28).

TABLE 28
CXS-744 gene expression in L6 treated with insulin for 6 hours

| | |
|---|---|
| Group | Relative Gene Expression (±SEM) |

| | | |
|---|---|---|
| 0 | nM Insulin | 101.82 ± 7.94 |
| 0.1 | nM Insulin | 132.47 ± 11.41 |
| 1 | nM Insulin | 143.97 ± 10.27 |
| 10 | nM Insulin | 152.56 ± 13.56 |
| 100 | nM Insulin | 199.91 ± 12.31 |
| 1000 | nM Insulin | 213.00 ± 10.61 |

### CXS-744 Protein is Detected in Media of L6 Muscle Cells

CXS-744 protein expression was analyzed in aliquots of media taken from differentiating L6 muscle cells in culture. Supernatants from these cultures were taken on successive days (days 1-8) post induction of differentiation and CXS-744 protein levels in was determined by Western blot. Densitometry of the protein band visible by Western blot indicates that media from L6 muscle cells contains CXS-744 protein by day 6. CXS-744 protein levels in the culture media continue to increase with levels at days 7 and 8 higher than those compared to day 6. This data clearly demonstrates that CXS-744 is a secreted protein and may represent a novel cytokine secreted from muscle.

TABLE 29
CXS-744 protein expression in culture
media taken from L6 muscle cells

| Group | Relative protein expression |
|---|---|
| Media from L6 muscle cells at Day 6 | 1.00 |
| Media from L6 muscle cells at Day 7 | 2.58 |
| Media from L6 muscle cells at Day 8 | 2.85 |

CXS-744 Gene Expression as Measured by SYBR Green Real Time PCR in P. obesus Mesenteric Fat

CXS-744 gene expression in mesenteric fat of NGT, IGT and T2D P. obesus in both the fed and fasted state was analyzed by real time PCR. In the NGT animals expression of CXS-744 was similar in both fed and fasted animals. In contrast, in both the IGT and T2D animals there was a tendency for CXS-744 gene expression to be lower in fasted compared with fed animals however the decrease was not significant.

In fed animals, CXS-744 expression was significantly higher in both the IGT and T2D animals compared to NGT animals (p=0.011 and 0.029 respectively). There was a strong positive correlation between CXS-744 gene expression in fed animals and bodyweight (p=0.015) whilst log insulin levels in fed animals were close to being significantly correlated with CXS-744 expression (p=0.054).

In fasted NGT, IGT and T2D P. obesus, there were no significant differences in CXS-744 gene expression in mesenteric fat. There was a significant correlation between CXS-744 expression and log insulin levels (p=0.044).

TABLE 30
CXS-744 gene expression in P. obesus mesenteric fat

| Group | Relative gene expression (±SEM) |
|---|---|
| NGT fed | 0.75 ± 0.16 |
| NGT fasted | 0.85 ± 0.17 |
| IGT fed | 2.11 ± 0.37 |
| IGT fasted | 1.48 ± 0.28 |
| T2D fed | 1.89 ± 0.40 |
| T2D fasted | 1.03 ± 0.22 |

CXS-744 Expression as Measured by SYBR Green Real Time PCR in P. obesus Subscapular Fat

CXS-744 gene expression in subscapular fat of fed NGT, IGT and T2D P. obesus was analyzed by real time PCR. In contrast to CXS-744 gene expression data obtained from mesenteric adipose tissue, CXS-744 gene expression in subscapular fat was significantly higher in IGT and T2D animals compared to NGT (p<0.001 in both cases) and was strongly correlated with bodyweight (p<0.001), % body fat (p<0.001), log glucose (p=0.049) and log insulin (p<0.001). This data identifies CXS-744 as a new adipocytokine that more highly expressed in the adipose tissue of IGT and T2D compared with nGT P. obesus. This data raises the possibility that CXS-744 may contribute to the development of insulin resistance and dysregulation of glucose homeostasis associated with type 2 diabetes.

TABLE 31
CXS-744 gene expression in P. obesus subscapular fat

| Group | Relative gene expression (±SEM) |
|---|---|
| NGT fed | 1.05 ± 0.12 |
| IGT fed | 3.38 ± 0.31 |
| T2D fed | 3.60 ± 0.23 |

CXS-744 Protein Expression in Different Tissues of P. obesus as Measured by Western Blot Analysis

The tissue distribution of CXS-744 protein was analyzed by Western blot. Western blots of three separate sets of tissues were performed and then densitometry was used to estimate the expression of CXS-742 on each individual blot. The brain tissue lysate was used as a reference in all 3 blots. The expression of CXS-744 protein was strongest in adrenal, testes and liver tissue whilst other tissues displayed lower levels of CXS-744.

TABLE 32
CXS-744 protein expression in P. obesus tissue lysates

| Tissue | Relative protein expression |
|---|---|
| Western Blot 1 | |
| Liver | 1.55 |
| Heart | 0.63 |
| Kidney | 0.5 |
| Pancreas | 0.7 |
| Spleen | 0.65 |
| Testes | 1.42 |
| Adrenal | 2.82 |
| Brain | 1.00 |
| Western Blot 2 | |
| Peri-renal fat | 2.04 |
| Mesenteric fat | 1.19 |
| Subscapular fat | 0.98 |
| Epididymal fat | 1.95 |
| IM fat | 1.94 |
| Stomach | 1.44 |
| Small intestine | 1.09 |
| Brain | 1.00 |
| Western blot 3 | |
| Soleus muscle | 0.62 |
| Red gastrocnemius muscle | 0.16 |
| White gastrocnemius muscle | 0.28 |
| Plantaris muscle | 0.35 |
| EDL | 0.19 |
| Brain | 1.00 |

Plasma CXS-744 are measured by ELISA and found to be significantly elevated. This would suggest that circulating plasma CXS-744 may contribute to insulin resistance and therapeutic agents designed to alter CXS-744 levels or that modulate CXS-744 activity and in particular which down-regulate levels or activities are proposed to be useful therapies in the treatment of type 2 diabetes and related disorders.

In addition, elevated circulating levels of CXS-744 can also be used to predict the onset of diabetes and conditions associated therewith, such as blindness, nephropathy and/or cardiovascular disease as well as inflammation.

### EXAMPLE 13

### CXS-745

Sequence of Psammomys obesus CXS-745

The nucleotide sequence identified is shown in FIG. 6 (SEQ ID NO:6).

### CXS-745 Sequence Homology

Analysis of the P. obesus CXS-740 sequence revealed it was homologous to the Putative MAPK activating protein PM20, PM21 gene. Alternate names for this gene and its product are DKFZP566C0424, hypothetical protein DKFZP566C0424, Erato Doi 22, DNA segment, Chr 4, ERATO Doi 22, expressed (D4Ertd22e) gene.

### CXS-745 Literature Review and Bioinformatics

The protein encoded by this gene comprises 133 amino acids. It is not predicted to have a signal peptide but is predicted to be secreted by the SecretomeP program.

Mitogen-activated protein kinases (MAPKs) are components of sequential kinase cascades that are activated in response to a variety of extracellular signals (Gerwins et al, J Biol Chem 272 (13):8288-9295, 1997).

This gene is mapped to chromosome 1 (1p36.13). The function of the protein is unknown.

CXS-745 Gene Expression as Measured by SYBR Green Real Time PCR in the P. obesus Skeletal Muscle

CXS-745 gene expression in the red gastrocnemius muscle of NGT, IGT and T2D P. obesus, in the fed and fasted state, was analyzed by real time PCR. CXS-745 gene expression was significantly correlated with blood glucose concentration in the fasted state (p=0.049) and percent body fat (p=0.039) in the fed state (Table 33).

Therefore, increased production of CXS-745 was associated with subphenotypes related to obesity and type 2 diabetes in this animal model.

TABLE 33
CXS-745 gene expression in P. obesus skeletal muscle

| Group | Relative Gene Expression (±SEM) |
|---|---|
| NGT fed | 11.00 ± 1.97 |
| NGT fasted | 4.66 ± 0.95 |
| IGT fed | 9.84 ± 0.59 |
| IGT fasted | 6.37 ± 1.02 |
| T2D fed | 7.65 ± 0.55 |
| T2D fasted | 6.35 ± 0.15 |

### CXS-745 Gene Expression in Different Tissues as Measured by SYBR Green Real Time PCR

The CXS-745 gene was found to be expressed in all tissues examined in P. obesus, but was highest in the skeletal muscle, heart and lung (Table 34).

TABLE 34
CXS-745 Tissue Distribution

| Tissue | Relative Gene Expression |
|---|---|
| Hypothalamus | 1.00 |
| Cortex | 0.69 |
| Cerebellum | 1.57 |
| Hippocampus | 0.84 |
| Midbrain | 1.26 |
| Brainstem | 1.20 |
| Liver | 3.11 |
| Red Gastrocnemius Muscle | 7.59 |
| White Gastrocnemius Muscle | 5.78 |
| Plantaris | 5.37 |
| EDL | 6.70 |
| Soleus muscle | 2.60 |
| Subscapular Fat | 2.27 |
| Intramuscular Fat | 2.19 |
| Epididymal Fat | 2.16 |
| Mesenteric Fat | 3.57 |
| Peri-renal Fat | 3.41 |
| Heart | 4.11 |
| Stomach | 1.95 |
| Kidney | 2.23 |
| Spleen | 2.20 |
| Adrenal | 3.56 |
| Large Intestine | 0.44 |
| Small Intestine | 1.79 |
| Lung | 6.50 |
| Testes | 1.32 |
| Ovary | 2.50 |

CXS-745 Gene Expression in L6 Muscle Cells Treated with Insulin as Measured by SYBR Green Real Time PCR

L6 cells were treated with 0, 0.1 nM, 1 nM, 10 nM, 100 nM or 1000 nM insulin for 6 hrs. Real time PCR showed that CXS-745 gene expression was significantly increased following treatment with 1 nM (p=0.008), 10 nM (p=0.008), 100 nM (p=0.001) and 1000 nM (p=0.001) insulin (Table 35)

TABLE 35
CXS-745 gene expression in L6 treated with insulin for 6 h

| | |
|---|---|
| Group | Relative Gene Expression (±SEM) |

| | | |
|---|---|---|
| 0 | nM Insulin | 101.30 ± 7.08 |
| 0.1 | nM Insulin | 118.04 ± 7.53 |
| 1 | nM Insulin | 132.75 ± 8.69 |
| 10 | nM Insulin | 132.47 ± 8.49 |
| 100 | nM Insulin | 144.33 ± 6.95 |
| 1000 | nM Insulin | 141.14 ± 7.99 |

### BIBLIOGRAPHY

Abdel-Wahab et al, Biochem J 362 (3):643-649, 2002
Alanen et al, J Biol Chem 278 (31):28912-28920, 2003
Altschul et al., Nucl. Acids Res. 25: 3389, 1997
Ameye and Young, Glycobiology 12 (9):107R-116R, 1999
Ausubel et al., “Current Protocols in Molecular Biology” John Wiley & Sons Inc, 1994-1998, Chapter 15
Bao et al, Cancer Cell 5 (4):329-339, 2004
Bonner and Laskey, Eur. J. Biochem. 46: 83, 1974
Calvo et al, J. Biol. Chem. 266 (17):11237-11244, 1991
Christodoulou; American Journal of Medical Genetics; 50 (3):255-64, 1994
Danielson et al, Genomics 15 (1):146-160, 1993
Gerwins et al, J Biol Chem 272 (13):8288-9295, 1997
Knocblach et al, Mol Cell Proteomics 2 (10):1104-1119, 2003
Knoll et al, Physiol Genomics, 21 (2):222-229, 2005
Koo, et. al.; Annals of Neurology; 34 (1)25-32, 1993
Liu et al, Gene 315:71-78, 2003
Luft; Proceedings of the National Academy of Sciences of the United States of America; 91 (19):8731-8, 1994
Marmur and Doty, J. Mol. Biol. 5: 109, 1962
Mogyorosi and Ziyadeh, Nephrol Dial Transplant 14 (5):1078-1081, 1999
Musil et al, J Cell Biol 114 (4):821-826, 1991
Olguin et al, Dev Biol 259 (2):209-224, 2003
Olsson et al, Diabetes 48 (3):616-622, 1999
Oshima et al, J Cell Biochem 96 (4):792-804, 2002
Schaefer et al, Faseb J 15 (3):559-561, 2001
Strazynski et al, J. Biol. Chem. 279 (20: 21266-21270, 2004

1. A method for the prognosis of diabetes or a complication arising from diabetes in a subject, said method comprising screening for elevated levels of CXS-741 (SEQ ID NO:2) and/or CXS-744 (SEQ ID NO: 5) protein or mRNA encoding said protein or a homolog thereof in a biological sample from said subject wherein an elevated level is indicative of diabetes or a complication arising therefrom or a likelihood of development of same.

2. The method of claim 1 wherein an elevated level of CXS-741 or its mRNA or a homolog thereof is detected.

3. The method of claim 1 wherein an elevated level of CXS-744 or its mRNA or a homolog thereof are detected.

4. The method of claim 1 wherein an elevated level of CXS-741 and CXS-744 or their mRNA or a homologs thereof is detected.

5. The method of claim 1 wherein the subject is a human and a human homolog of CXS-741 and/or CXS-744 is detected.

6. The method of claim 1 wherein the complication of diabetes is blindness, nephropathy and/or cardiovascular disease.

7. The method of claim 1 wherein the biological sample is whole blood, blood plasma and/or serum.

8. A method for the prognosis of an inflammatory condition in a subject, said method comprising screening for elevated levels of CXS-741 (SEQ ID NO:2) and/or CXS-744 (SEQ ID NO: 5) protein or mRNA encoding said protein or a homolog thereof in a biological sample from said subject wherein an elevated level is indicative of an inflammatory condition.

9. The method of claim 8 wherein an elevated level of CXS-741 or its mRNA or a homolog thereof is detected.

10. The method of claim 8 wherein an elevated level of CXS-744 or its mRNA or a homolog thereof is detected.

11. The method of claim 8 wherein an elevated level of CXS-741 and CXS-744 or their mRNA or a homolog thereof is detected.

12. The method of claim 8 wherein the subject is a human.

13. The method of claim 8 wherein the inflammatory condition is selected from acne, angina, arthritis, aspiration pneumonia, disease, empyema, gastroenteritis, inflammation, intestinal flu, NEC, necrotizing enterocolitis, pelvic inflammatory disease, pharyngitis, PID, pleurisy, raw throat, redness, rubor, sore throat, stomach flu, urinary tract infection, Chronic Inflammatory Demyelinating Polyneuropathy and Chronic Inflammatory Demyelinating Polyradiculoneuropathy.

14. The method of claim 8 wherein the biological sample is whole blood, blood plasma and/or serum.

15. A method of modulating expression of one or more of CXS-741 (SEQ ID NO:2), and/or CXS-744 (SEQ ID NO: 5) in a mammal, said method comprising contacting CXS-741 and/or CXS-744 with an effective amount of an agent capable of modulating CXS-741 and/or CXS-744 expression for a time and under conditions sufficient to up-regulate or down-regulate or otherwise modulate expression CXS-741 and/or CXS-744.

16. A method of modulating activity of one or more of CXS-741 (SEQ ID NO:2) and/or CXS-744 (SEQ ID NO: 5) in a mammal, said method comprising administering to said mammal an effective amount of an agent capable of modulating the activity of one or more of CXS-741 and/or CXS-744 for a time and under conditions sufficient to increase or decrease or otherwise modulate the activity of one or more of CXS-741 and/or CXS-744.

17. A method of treating a subject suffering from diabetes or a complication thereof, said method comprising administering to said subject an effective amount of an agent for a time and under conditions sufficient to down-regulate the level or activity CXS-741 (SEQ ID NO:2) and/or CXS-744 (SEQ ID NO:5).

18. The method of claim 17 wherein the complication of diabetes is blindness, nephropathy and/or cardiovascular disease.

19. The method of claim 17 wherein the subject is human.

20. A method of treating a subject suffering from an inflammatory condition said method comprising administering to said subject an effective amount of an agent for a time and under conditions sufficient to down-regulate the level of activity of CXS-741 (SEQ ID NO:2) and/or CXS-744 (SEQ ID NO:5).

21. The method of claim 20 wherein the inflammatory condition is acne, angina, arthritis, aspiration pneumonia, disease, empyema, gastroenteritis, inflammation, intestinal flu, NEC, necrotizing enterocolitis, pelvic inflammatory disease, pharyngitis, PID, pleurisy, raw throat, redness, rubor, sore throat, stomach flu, urinary tract infection, Chronic Inflammatory Demyelinating Polyneuropathy and Chronic Inflammatory Demyelinating Polyradiculoneuropathy.

22. The method of claim 20 wherein the subject is human.

23. A method for identifying a nucleic acid molecule comprising a signal sequence which facilitates export of a cytokine out of a cell, said method comprising generating a cDNA library and inserting DNA fragments into a vector upstream of a genetic sequence encoding a cytokine such that upon expression, the inserted DNA fragment encodes a molecule operably fused to said cytokine, transfecting a cell line dependent on said cytokine for survival and screening for live cells wherein live cells is indicative of a DNA fragment encoding a signal sequence wherein DNA fragments which enable transport of the cytokine comprise a nucleotide sequence selected from the list consisting of:
(i) a nucleotide sequence as set forth in SEQ ID NO:2 (CXS-741) or a nucleotide sequence having at least about 90% identity thereto or a nucleotide sequence capable of hybridizing to SEQ ID NO:2 (CXS-741) or its complementary form; and
(ii) a nucleotide sequence as set forth in SEQ ID NO: 5 (CXS-744) or a nucleotide sequence having at least about 90% identity thereto or a nucleotide sequence capable of hybridizing to SEQ ID NO: 5 (CXS-744) or its complementary form.

24. The method of claim 23 wherein the cytokine is IL-3.

25. The method of claim 23 wherein the DNA fragment is derived from P. obesus or a human homology thereof.

26. A nucleic acid molecule identified by the method of claim 23.

27. The nucleic acid molecule of claim 26 wherein the nucleic acid molecule comprises the nucleotide sequence set forth in SEQ ID NO:2 (CXS-741).

28. The nucleic acid molecule of claim 26 wherein the nucleic acid molecule comprises the nucleotide sequence set forth in SEQ ID NO: 5 (CXS-744).

29. Use of the nucleic acid molecule of claim 26 in the manufacture of a medicament for the treatment of diabetes, a complication therefrom or an inflammatory condition.


SEQUENCE LISTING (11 sequences)

SEQ ID NO: 1 - DNA, 685 bp, Psammomys obesus
  misc_feature (17)..(17): n is a, c, g, or t
gcagactcgggacccancatgaagctggccctcctgccttngattntgatgntgcnggcgacagtcccaggccccaggtgcatagcaggtgcccaggccagctgctccntgagatgcggcgaacaggatggactntgctcttgtcatccaacctgntcgggcctnggtacctgttgtgaagactttntgagctactgcctggagatttcaccctcctcaggntccatgatgggtggcaaagactttgtggtgcagcatttaaagtgnccttactccaccgatggcgtcatctgcaggtttaaggagagtatccagacccttgggtatgtcgacagcttgaaccaagtgcactgtgtatcacccttgctctatgaaagcggccaagttcccttcactgtatcgttggacaatggccgctccttccttcgagcgggcacttggctggccgctcatccctacaaagtatccgagtccgagaagagccagctggagaatgagacctactggcaatactatggcacctcagacacgatgggaaacctcagcctcacctggaacatctccatgctgcccatgccggccgtcaccattgagctttggggctatgaggagacagggaaaccctattctgagaactggacagcaaagtggtcctatctataccctctggcgacagacatcccca

SEQ ID NO: 2 - DNA, 587 bp, Psammomys obesus
  misc_feature (27)..(27): n is a, c, g, or t
acccggggatcagatctcgacccacgngtccgagggctcctgtggcaaatacccggattaaaaggtttccaggttgtgacattacatgatacaatcatgaaggcaactctcattttactccttctggcacaagtctcttgggctggaccatttgaacagagaggcttatttgacttcatgctagaagatgaggcttctgggatgagcgaggaagaccagcgtcttcctccgcctgatctaccagacgtgatgcccctggtacccgagtgtcccttcagctgccagtgtcatcttcgagtggtgcagtgttctgatttgggtatggcaacagtgcccaagaaccttccatntgatacaacattgctagacctgcagaacaacaaaataactgagatcaaagatggggacttcaagaacctgaagaacttgcataccttgattcttgtcaacaacaagatcagcaagatcagccccggagcgttcacacctctagtgaaattggaacggctttatctgtctaagaaccgactgaaggaactgcccgaaaaaatgcccaaaacgnggcaggagcttcgcgcccatgagaa

SEQ ID NO: 3 - DNA, 446 bp, Psammomys obesus
  misc_feature (11)..(11): n is a, c, g, or t
tagcgctaccnggantcagatctcgacccacgcgtccggagctcagggntgaagatgattcctctcctgcccttatctactntgntgctccttntcctgntgtgtgaccttaaccctgcaaatgccaacagttantatgacaaggtcctggctcacagccgcatcaggggccgggatcagggcccaaatgtctgtgccctccagcagatcctgggcaccaaaaagaaatacttcagctcctgtaagaactggtatcaaggtgctatctgcgggaagaaaaccantgtgttatacgaatgttgtcctggctatatgagaatggaaggaatgaaaggctgcccggcagtgatgcctatcgaccatgtttanggcactctgggcattgtgggagccaccaccactcagcactattccgatgtctccaagggagagaagagatgaagaaa

SEQ ID NO: 4 - DNA, 452 bp, Psammomys obesus
  misc_feature (18)..(18): n is a, c, g, or t
tcagatctcgacccacgngtcngngtacggagagagagggctcgcgcgccgggaagaagcggagagcttccccgatggtgccgccgcctcctctgagccggggaggagcagccaaggggcagctgggcaagagcctggggccgctgctactactcctggngctgggacacacgtggncctaccgagaggaacccgaggaccgcgacagagaagtgtgctcggaaaacaagatcaccacgaccaaatacccgtgtctgaagtcttcgggcgagctcaccacgtgcttcaggaaaaaatgctgtaaaggatataaatttgttcttggacagtgtattccagaagattatgacatctgtgcccaagccccctgcgaacagcaatgcacggataacttcggccgagttctgtgcacgtgttagccccgggacngatatgaccgcgagagacncctc

SEQ ID NO: 5 - DNA, 641 bp, Psammomys obesus
  misc_feature (109)..(109): n is a, c, g, or t
gcgtcgaaaaaccaatctgggcacccggggggcccggctgagcggcggtgcggcgccacctcctcgcgcggcggcccggagcagccgcctccgccgagatgtcgcctcncctcggggccacctgttggctgggcttcggtctcctgctcctcgtcgctccttccgaggggcgcgtcgggctcggaaagggttttggagaccatattcactggaggactctcgaagatgggaagaaagaggcagccgcaagtggcttgccgctgatggtgatcatccataagtcttggtgtggagcatgcaaagctttaaaacctaaatttgcagaatctacagaaatttcagaactgtcccataattttgttatggtaaatctggaggatgaagaggaacccaaggatgaagatttcagtcccgatgggggttatatcccacggatccttttcttggatcccagtggcaaggtgcgccctgnaaatcatcaatgagagtggaaaccccagctacaaatatttctacgtcagngctgancaagttgttcaagggatgaaggaagctcagnanaggctgaccggcnatgccttcananacaaanacctncaaaatganaaggggcggccncaagcttcaatcagnggccgaaa

SEQ ID NO: 6 - DNA, 468 bp, Psammomys obesus
  misc_feature (29)..(29): n is a, c, g, or t
ctgagagctgggaggaggcggcagacagngggcaggaaatccaaatctcctcccaaagtgcccattgtgattcaagacgatagccttcccacgggcccccctccacagatccgcatcctcaagaggcccaccagcaacggtgtggtcagcagcccccaactccaccaacaggccagcccttcctgtcaagtccctcgcacagcgggaggcagagtacgcagaggcccggagaaggatcctgggcagttccagccctgaggaggaacaggagaaacccatcctcgacaggccaaccaggatctctcaacctgaagacagcaggcagcccagtaatgtgatcagacagcctttgggtcctgacgggtcacaaggcttcaaacagcgcagataaatgcggacaagaaaggatgccgctgccaccgctgccgccaccatcatcgcctcgtggattgtctgccatgggttgca

SEQ ID NO: 7 - DNA, 31 bp, Artificial Sequence
  primer Not1
tctagatcgcgagcggccgcccnnnnnnnnn

SEQ ID NO: 8 - DNA, 23 bp, Artificial Sequence
  forward PCR primer
ctggtttagtgaaccgtcagatc

SEQ ID NO: 9 - DNA, 22 bp, Artificial Sequence
  reverse PCR primer
ctccttgacaatagagctgcaa

SEQ ID NO: 10 - DNA, 20 bp, Artificial Sequence
  forward PCR primer
tagcgctaccggactcagat

SEQ ID NO: 11 - DNA, 19 bp, Artificial Sequence
  reverse PCR primer
cggccactgattgaagctt